(19) United States
(12) Patent Application Publication
(10) Pub. No.: US 2026/0083320 A1
(43) Pub. Date: Mar. 26, 2026

(54) DETECTION OF DRY-EYE

(71) Applicant: Apple Inc., Cupertino, CA (US)

(72) Inventors: Irene CHEN, San Jose, CA (US); Emmanuel B. ALABI, San Diego, CA (US)

(21) Appl. No.: 19/323,974

(22) Filed: Sep. 9, 2025

Related U.S. Application Data
(60) Provisional application No. 63/699,785, filed on Sep. 26, 2024.

(51) Int. Cl. A61B 3/10; A61B 3/00; A61B 3/14

(52) U.S. Cl. CPC A61B 3/101; A61B 3/0025; A61B 3/005; A61B 3/14

(57) ABSTRACT

An electronic device obtains, via the one or more sensors, data including one or more images of one or both eyes. One or more features can be extracted for one or both eyes of a user of the electronic device. In accordance with a determination that one or more one or more criteria are satisfied, including at least once criterion based on the one or more features of one or both eyes of the user, the electronic device determines a dry-eye condition associated with one or both eyes of the user. In some examples, in accordance with a determination that a dry-eye condition is associated with one or both eyes of the user, the electronic device provides one or more mitigations.

### CROSS-REFERENCE TO RELATED APPLICATIONS

This application claims the benefit of U.S. Provisional Application No. 63/699,785, filed Sep. 26, 2024, the content of which is herein incorporated by reference in its entirety for all purposes.

### FIELD OF THE DISCLOSURE

This relates generally to systems and methods for monitoring one or both eyes, and more specifically, to determining and mitigating a dry-eye condition for a user of an electronic device.

### BACKGROUND OF THE DISCLOSURE

The use of wearable computing devices has increased recently. Some wearable computing devices take images of the eyes using cameras and use the images to track the direction the eyes are looking.

### SUMMARY OF THE DISCLOSURE

Described herein are systems and methods for using sensor data (such as thermal images) of one or both eyes of a user of an electronic device to determine and/or mitigate a dry-eye condition. The electronic device can use one or more criteria, including at least one criterion that is based on sensor data of one or both eyes. The satisfaction of the one or more criteria can be used, in some examples, to determine a dry-eye condition of the user. In some examples, an electronic device (e.g., a head-mounted device) includes one or more image sensors, including one or more image sensors that are positioned to image one or more of the eyes of the user of the electronic device. In some examples, one or more features can be extracted from the sensor data. In one or more examples, extracting spatial properties of one or both eyes of the user includes segmenting the sensor data to identify the one or more regions of one or both eyes of the user. In one or more examples, in accordance with a determination that one or more criteria are satisfied, the electronic device provides an indication of a dry-eye condition and/or provides one or more dry-eye mitigations to the user of the electronic device.

In one or more examples, as part of the extracted features of one or both eyes of the user, the electronic device determines spatial properties of one or both eyes of the user of the electronic device. In one or more examples, the electronic device segments the extracted features of the one or both eyes of the user to identify various regions and features of the eye. In one or more examples, the electronic device determines a cooling rate of one or both eyes of the user and uses the cooling rate to satisfy one or more criteria. In one or more examples, when the electronic device determines that the one or more criteria are satisfied, the electronic device determines a risk of dry-eye condition associated with one or both eyes of the user of the electronic device. In one or more examples, the electronic device notifies the user of a possible dry-eye condition and/or suggests mitigations or follow-up for diagnosis with a health care provider. In one or more examples, the electronic device utilizes fans to modify blinking behavior of the user to prevent or mitigate a possible dry-eye condition.

The full descriptions of these examples are provided in the Drawings and the Detailed Description, and it is understood that this Summary does not limit the scope of the disclosure in any way.

### BRIEF DESCRIPTION OF THE DRAWINGS

For improved understanding of the various examples described herein, reference should be made to the Detailed Description below along with the following drawings. Like reference numerals often refer to corresponding parts throughout the drawings.

FIG. 1 illustrates an electronic device presenting an extended reality environment according to some examples of the disclosure.

FIG. 2 illustrates a block diagram of an example architecture for a device according to some examples of the disclosure.

FIG. 3 illustrates an example electronic device configured to determine and mitigate a dry-eye condition of one or both eyes of the user according to some examples of the disclosure.

FIG. 4 is a flow diagram illustrating a method for determining and mitigating a dry-eye condition based on extracted features according to some examples of the disclosure.

FIG. 5 is a flow diagram illustrating a method for processing images of one or both eyes of a user of an electronic device according to some examples of the disclosure.

FIG. 6 illustrates a representation of features extracted from a normal eye and a red eye of a user of an electronic device according to some examples of the disclosure.

FIG. 7 is a flow diagram illustrating a method for determining eye redness using the one or more extracted features according to some examples of the disclosure.

FIG. 8 illustrates a representation of features extracted from a non-vasodilated eye and a vasodilated eye of a user of an electronic device according to some examples of the disclosure.

FIG. 9 is a flow diagram illustrating a method for determining blood vessel dilation from the one or more extracted features according to some examples of the disclosure.

FIG. 10 illustrates a representation of features extracted from a normal tear film and a dry-eye tear film associated with one or both eyes of a user of an electronic device according to some examples of the disclosure.

FIG. 11 is a flow diagram illustrating a method for determining spatial properties from the one or more extracted features according to some examples of the disclosure.

FIG. 12 illustrates a representation of temperature data at various regions of interest associated with one or both eyes of a user of an electronic device according to some examples of the disclosure.

FIG. 13 is a flow diagram illustrating a method for determining a cooling rate from the extracted temperature data according to some examples of the disclosure.

FIG. 14 is an example plot illustrating a cooling rate of one or both eyes of a user of an electronic device by temperature and time according to some examples of the disclosure.

FIG. 15 is a flow diagram illustrating a method for applying one or more machine learning models to determine a dry-eye condition according to some examples of the disclosure.

FIG. 16 illustrates a representation of a notification displayed by an electronic device according to some examples of the disclosure.

FIG. 17 is a flow diagram illustrating a method for displaying a notification as a mitigation according to some examples of the disclosure.

FIG. 18 is a flow diagram illustrating a method for a fan mitigation according to some examples of the disclosure.

FIG. 19 is a flow diagram illustrating a method for determining a dry-eye condition according to some examples of the disclosure.

### DETAILED DESCRIPTION

Described herein are systems and methods for using sensor data (such as thermal images) of one or both eyes of a user of an electronic device to determine and/or mitigating a dry-eye condition. The electronic device can use one or more criteria, including at least one criterion that is based on sensor data of one or both eyes. The satisfaction of the one or more criteria can be used, in some examples, to determine a dry-eye condition of the user. In some examples, an electronic device (e.g., a head-mounted device) includes one or more sensors, including one or more image sensors that are positioned to image one or more of the eyes of the user of the electronic device. In some examples, one or more features can be extracted from the sensor data. In one or more examples, extracting properties of one or both eyes of the user includes segmenting the sensor data to identify one or more regions of one or both eyes of the user. In one or more examples, in accordance with a determination that one or more criteria are satisfied, the electronic device provides an indication of a dry-eye condition and/or provides one or more dry-eye mitigations to the user of the electronic device.

In one or more examples, as part of the extracting features of one or both eyes of the user, the electronic device determines spatial properties of one or both eyes of the user of the electronic device. In one or more examples, as part of extracting the features of one or both eyes of the user, the electronic device segments the sensor data of one or both eyes of the user to identify various regions of the eye. In one or more examples, the electronic device determines a cooling rate of one or both eyes of the user and uses the cooling rate to satisfy one or more criteria. In one or more examples, when the electronic device determines that the one or more criteria are satisfied, the electronic device determines a dry-eye condition associated with one or both eyes of the user of the electronic device. In one or more examples, the electronic device notifies the user of a possible dry-eye condition and/or suggests mitigations or follow-up for diagnosis with a health care provider. In one or more examples, the electronic device utilizes fans to modify blinking behavior of the user to prevent or mitigate a possible dry-eye condition.

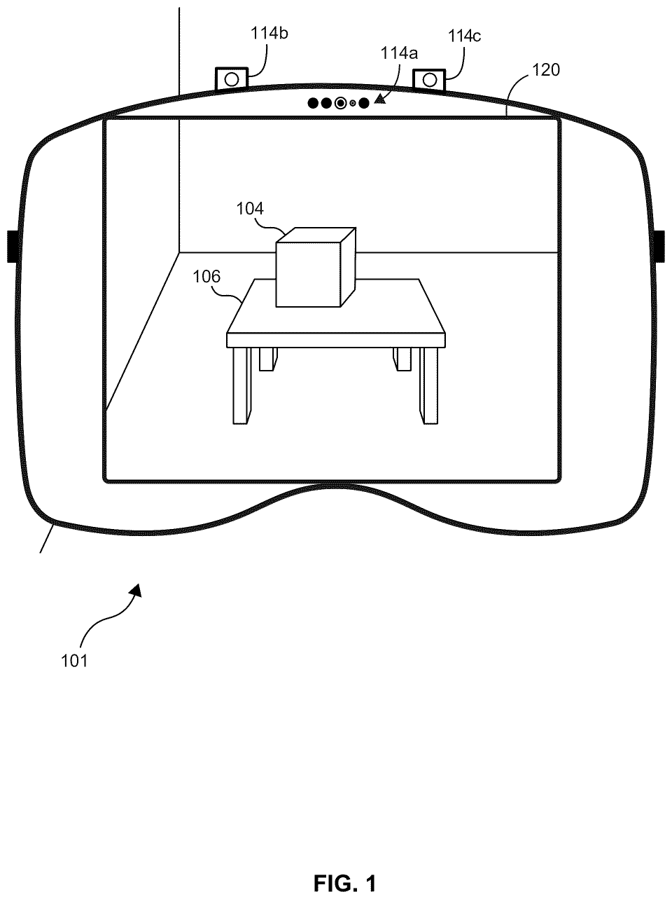

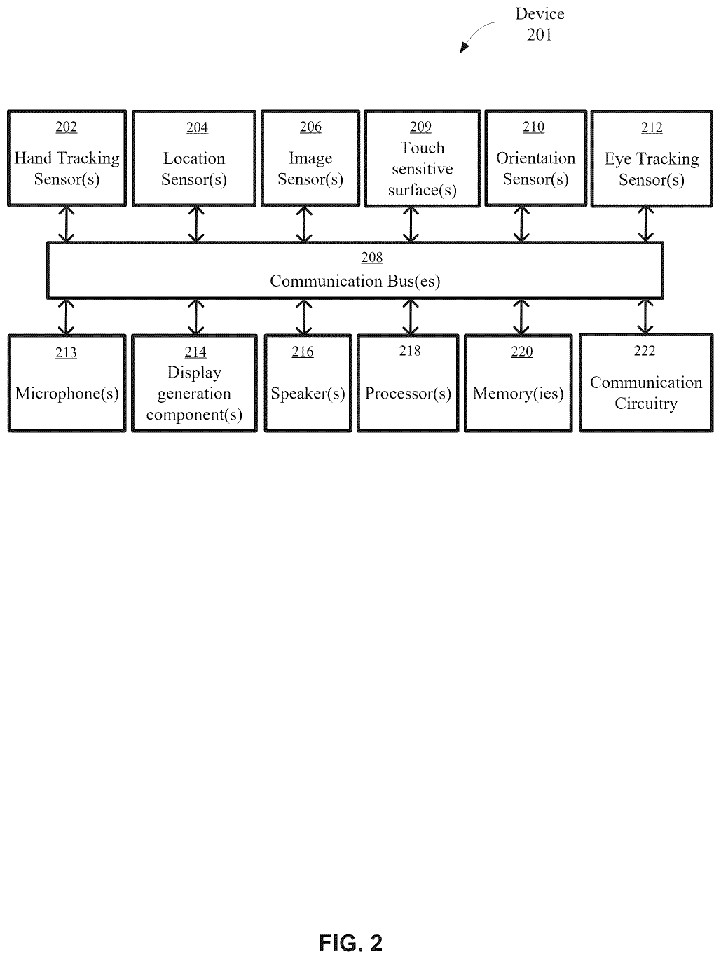

FIG. 1 illustrates an electronic device 101 presenting an extended reality (XR) environment (e.g., a computer-generated environment optionally including representations of physical and/or virtual objects) according to some examples of the disclosure. In some examples, as shown in FIG. 1, electronic device 101 is a head-mounted display or other head-mountable device configured to be worn on a head of a user of the electronic device 101. Examples of electronic device 101 are described below with reference to the architecture block diagram of FIG. 2. As shown in FIG. 1, electronic device 101 and table 106 are located in a physical environment. The physical environment may include physical features such as a physical surface (e.g., floor, walls) or a physical object (e.g., table, lamp, etc.). In some examples, electronic device 101 may be configured to detect and/or capture images of physical environment including table 106 (illustrated in the field of view of electronic device 101).

In some examples, as shown in FIG. 1, electronic device 101 includes one or more internal image sensors 114a oriented towards a face of the user (e.g., eye tracking cameras described below with reference to FIG. 2). In some examples, internal image sensors 114a are used for eye tracking (e.g., detecting a gaze of the user). Internal image sensors 114a are optionally arranged on the left and right portions of display 120 to enable eye tracking of the user's left and right eyes. In some examples, electronic device 101 also includes external image sensors 114b and 114c facing outwards from the user to detect and/or capture the physical environment of the electronic device 101 and/or movements of the user's hands or other body parts.

In some examples, display 120 has a field of view visible to the user (e.g., that may or may not correspond to a field of view of external image sensors 114b and 114c). Because display 120 is optionally part of a head-mounted device, the field of view of display 120 is optionally the same as or similar to the field of view of the user's eyes. In other examples, the field of view of display 120 may be smaller than the field of view of the user's eyes. In some examples, electronic device 101 may be an optical see-through device, in which display 120 is a transparent or translucent display through which portions of the physical environment may be directly viewed. In some examples, display 120 may be included within a transparent lens and may overlap all or only a portion of the transparent lens. In other examples, electronic device may be a video-passthrough device, in which display 120 is an opaque display configured to display images of the physical environment captured by external image sensors 114b and 114c. While a single display 120 is shown, it should be appreciated that display 120 may include a stereo pair of displays.

In some examples, in response to a trigger, the electronic device 101 may be configured to display a virtual object 104 in the XR environment represented by a cube illustrated in FIG. 1, which is not present in the physical environment, but is displayed in the XR environment positioned on the top of real-world table 106 (or a representation thereof). Optionally, virtual object 104 can be displayed on the surface of the table 106 in the XR environment displayed via the display 120 of the electronic device 101 in response to detecting the planar surface of table 106 in the physical environment 100.

In some examples, the display 120 is provided as a passive component (e.g., rather than an active component) within electronic device 101. For example, the display 120 may be a transparent or translucent display, as mentioned above, and may not be configured to display virtual content (e.g., images of the physical environment captured by external image sensors 114b and 114c and/or virtual object 104). Alternatively, in some examples, the electronic device 101 does not include the display 120. In some such examples, in which the display 120 is provided as a passive component or is not included in the electronic device 101, the electronic device 101 may still include sensors (e.g., internal image sensor 114a and/or external image sensors 114b and 114c) and/or other input devices, such as one or more of the components described below with reference to FIG. 2.

It should be understood that virtual object 104 is a representative virtual object and one or more different virtual objects (e.g., of various dimensionality such as two-dimensional or other three-dimensional virtual objects) can be included and rendered in a three-dimensional XR environment. For example, the virtual object can represent an application or a user interface displayed in the XR environment. In some examples, the virtual object can represent content corresponding to the application and/or displayed via the user interface in the XR environment. In some examples, the virtual object 104 is optionally configured to be interactive and responsive to user input (e.g., air gestures, such as air pinch gestures, air tap gestures, and/or air touch gestures), such that a user may virtually touch, tap, move, rotate, or otherwise interact with, the virtual object 104.

In some examples, displaying an object in a three-dimensional environment may include interaction with one or more user interface objects in the three-dimensional environment. For example, initiation of display of the object in the three-dimensional environment can include interaction with one or more virtual options/affordances displayed in the three-dimensional environment. In some examples, a user's gaze may be tracked by the electronic device as an input for identifying one or more virtual options/affordances targeted for selection when initiating display of an object in the three-dimensional environment. For example, gaze can be used to identify one or more virtual options/affordances targeted for selection using another selection input. In some examples, a virtual option/affordance may be selected using hand-tracking input detected via an input device in communication with the electronic device. In some examples, objects displayed in the three-dimensional environment may be moved and/or reoriented in the three-dimensional environment in accordance with movement input detected via the input device.

In the discussion that follows, an electronic device that is in communication with a display generation component and one or more input devices is described. It should be understood that the electronic device optionally is in communication with one or more other physical user-interface devices, such as a touch-sensitive surface, a physical keyboard, a mouse, a joystick, a hand tracking device, an eye tracking device, a stylus, etc. Further, as described above, it should be understood that the described electronic device, display and touch-sensitive surface are optionally distributed amongst two or more devices. Therefore, as used in this disclosure, information displayed on the electronic device or by the electronic device is optionally used to describe information outputted by the electronic device for display on a separate display device (touch-sensitive or not). Similarly, as used in this disclosure, input received on the electronic device (e.g., touch input received on a touch-sensitive surface of the electronic device, or touch input received on the surface of a stylus) is optionally used to describe input received on a separate input device, from which the electronic device receives input information.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, a television channel browsing application, and/or a digital video player application.

FIG. 2 illustrates a block diagram of an example architecture for a device 201 according to some examples of the disclosure. In some examples, electronic device 201 includes one or more electronic devices. For example, the electronic device 201 may be a portable device, an auxiliary device in communication with another device, a head-mounted display, etc., respectively. In some examples, electronic device 201 corresponds to electronic device 101 described above with reference to FIG. 1.

As illustrated in FIG. 2, the electronic device 201 optionally includes various sensors, such as one or more hand tracking sensors 202, one or more location sensors 204, one or more image sensors 206 (optionally corresponding to internal image sensors 114a and/or external image sensors 114b and 114c in FIG. 1), one or more touch-sensitive surfaces 209, one or more motion and/or orientation sensors 210, one or more eye tracking sensors 212, one or more microphones 213 or other audio sensors, one or more body tracking sensors (e.g., torso and/or head tracking sensors), one or more display generation components 214, optionally corresponding to display 120 in FIG. 1, one or more speakers 216, one or more processors 218, one or more memories 220, and/or communication circuitry 222. One or more communication buses 208 are optionally used for communication between the above-mentioned components of electronic devices 201.

Communication circuitry 222 optionally includes circuitry for communicating with electronic devices, networks, such as the Internet, intranets, a wired network and/or a wireless network, cellular networks, and wireless local area networks (LANs). Communication circuitry 222 optionally includes circuitry for communicating using near-field communication (NFC) and/or short-range communication, such as Bluetooth®.

Processor(s) 218 include one or more general processors, one or more graphics processors, and/or one or more digital signal processors. In some examples, memory 220 is a non-transitory computer-readable storage medium (e.g., flash memory, random access memory, or other volatile or non-volatile memory or storage) that stores computer-readable instructions configured to be executed by processor(s) 218 to perform the techniques, processes, and/or methods described below. In some examples, memory 220 can include more than one non-transitory computer-readable storage medium. A non-transitory computer-readable storage medium can be any medium (e.g., excluding a signal) that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on compact disc (CD), digital versatile disc (DVD), or Blu-ray technologies, as well as persistent solid-state memory such as flash, solid-state drives, and the like.

In some examples, display generation component(s) 214 include a single display (e.g., a liquid-crystal display (LCD), organic light-emitting diode (OLED), or other types of display). In some examples, display generation component(s) 214 include multiple displays. In some examples, display generation component(s) 214 can include a display with touch capability (e.g., a touch screen), a projector, a holographic projector, a retinal projector, a transparent or translucent display, etc. In some examples, electronic device 201 includes touch-sensitive surface(s) 209, respectively, for receiving user inputs, such as tap inputs and swipe inputs or other gestures. In some examples, display generation component(s) 214 and touch-sensitive surface(s) 209 form touch-sensitive display(s) (e.g., a touch screen integrated with electronic device 201 or external to electronic device 201 that is in communication with electronic device 201).

Electronic device 201 optionally includes image sensor(s) 206. Image sensors(s) 206 optionally include one or more visible light image sensors, such as charged coupled device (CCD) sensors, and/or complementary metal-oxide-semiconductor (CMOS) sensors operable to obtain images of physical objects from the real-world environment. Image sensor(s) 206 also optionally include one or more infrared (IR) sensors, such as a passive or an active IR sensor, for detecting infrared light from the real-world environment. For example, an active IR sensor includes an IR emitter for emitting infrared light into the real-world environment. Image sensor(s) 206 also optionally include one or more cameras configured to capture movement of physical objects in the real-world environment. Image sensor(s) 206 also optionally include one or more depth sensors configured to detect the distance of physical objects from electronic device 201. In some examples, information from one or more depth sensors can allow the device to identify and differentiate objects in the real-world environment from other objects in the real-world environment. In some examples, one or more depth sensors can allow the device to determine the texture and/or topography of objects in the real-world environment.

In some examples, electronic device 201 uses CCD sensors, event cameras, and depth sensors in combination to detect the physical environment around electronic device 201. In some examples, image sensor(s) 206 include a first image sensor and a second image sensor. The first image sensor and the second image sensor work in tandem and are optionally configured to capture different information of physical objects in the real-world environment. In some examples, the first image sensor is a visible light image sensor and the second image sensor is a depth sensor. In some examples, electronic device 201 uses image sensor(s) 206 to detect the position and orientation of electronic device 201 and/or display generation component(s) 214 in the real-world environment. For example, electronic device 201 uses image sensor(s) 206 to track the position and orientation of display generation component(s) 214 relative to one or more fixed objects in the real-world environment.

In some examples, electronic device 201 includes microphone(s) 213 or other audio sensors. Electronic device 201 optionally uses microphone(s) 213 to detect sound from the user and/or the real-world environment of the user. In some examples, microphone(s) 213 include an array of microphones (a plurality of microphones) that optionally operate in tandem, such as to identify ambient noise or to locate the source of sound in space of the real-world environment.

Electronic device 201 includes location sensor(s) 204 for detecting a location of electronic device 201 and/or display generation component(s) 214. For example, location sensor(s) 204 can include a global positioning system (GPS) receiver that receives data from one or more satellites and allows electronic device 201 to determine the device's absolute position in the physical world.

Electronic device 201 includes orientation sensor(s) 210 for detecting orientation and/or movement of electronic device 201 and/or display generation component(s) 214. For example, electronic device 201 uses orientation sensor(s) 210 to track changes in the position and/or orientation of electronic device 201 and/or display generation component(s) 214, such as with respect to physical objects in the real-world environment. Orientation sensor(s) 210 optionally include one or more gyroscopes and/or one or more accelerometers.

Electronic device 201 includes hand tracking sensor(s) 202 and/or eye tracking sensor(s) 212 (and/or other body tracking sensor(s), such as leg, torso and/or head tracking sensor(s)), in some examples. Hand tracking sensor(s) 202 are configured to track the position/location of one or more portions of the user's hands, and/or motions of one or more portions of the user's hands with respect to the extended reality environment, relative to the display generation component(s) 214, and/or relative to another defined coordinate system. Eye tracking sensor(s) 212 are configured to track the position and movement of a user's gaze (eyes, face, or head, more generally) with respect to the real-world or extended reality environment and/or relative to the display generation component(s) 214. In some examples, hand tracking sensor(s) 202 and/or eye tracking sensor(s) 212 are implemented together with the display generation component(s) 214. In some examples, the hand tracking sensor(s) 202 and/or eye tracking sensor(s) 212 are implemented separate from the display generation component(s) 214.

In some examples, the hand tracking sensor(s) 202 (and/or other body tracking sensor(s), such as leg, torso and/or head tracking sensor(s)) can use image sensor(s) 206 (e.g., one or more IR cameras, 3D cameras, depth cameras, etc.) that capture three-dimensional information from the real-world including one or more body parts (e.g., hands, legs, or torso of a human user). In some examples, the hands can be resolved with sufficient resolution to distinguish fingers and their respective positions. In some examples, one or more image sensors 206 are positioned relative to the user to define a field of view of the image sensor(s) 206 and an interaction space, in which finger/hand position, orientation and/or movement captured by the image sensors are used as inputs (e.g., to distinguish from a user's resting hand or other hands of other persons in the real-world environment). Tracking the fingers/hands for input (e.g., gestures, touch, tap, etc.) can be advantageous in that it does not require the user to touch, hold or wear any sort of beacon, sensor, or other marker.

In some examples, eye tracking sensor(s) 212 include at least one eye tracking camera (e.g., infrared (IR) cameras) and/or illumination sources (e.g., IR light sources, such as LEDs) that emit light towards a user's eyes. The eye tracking cameras may be pointed towards a user's eyes to receive reflected IR light from the light sources directly or indirectly from the eyes. In some examples, both eyes are tracked separately by respective eye tracking cameras and illumination sources, and a focus/gaze can be determined from tracking both eyes. In some examples, one eye (e.g., a dominant eye) is tracked by one or more respective eye tracking cameras/illumination sources.

Electronic device 201 is not limited to the components and configuration of FIG. 2, but can include fewer, other, or additional components in multiple configurations. In some examples, electronic device 201 can be implemented between two electronic devices (e.g., as a system). In some such examples, each of the two (or more) electronic device may each include one or more of the same components discussed above, such as various sensors, one or more display generation components, one or more speakers, one or more processors, one or more memories, and/or communication circuitry. A person or persons using electronic device 201, is optionally referred to herein as a user or users of the device.

FIG. 3 illustrates an example head-mounted device (HMD) for implementing a dry-eye determination process according to some examples of the disclosure. In the example of FIG. 3, HMD 302 is another example electronic device similar to electronic device 101 described above with respect to FIGS. 1-2 above. In addition to the components of electronic device described above with respect to FIGS. 1-2, in one or more examples, HMD 302 further includes a thermal image sensor 304 and a visible image sensor 306 (e.g., corresponding to internal image sensors 114a, one or more eye tracking sensors 212, etc.). In one or more examples, thermal image sensor 304 and visible image sensor 306 are part of the image sensors 206 and/or one or more eye tracking sensors 212 described above with respect to FIG. 2. In one or more examples, both thermal image sensor 304 and visible image sensor 306 are disposed on HMD 302 such that they are directed toward the eyes of the user of the HMD 302. In some examples, thermal image sensor 304 is implemented as an infrared (IR) sensor that can obtain infrared images of one or both eyes of the user. In one or more examples, visible image sensor 306 is implemented as a camera that collects images of one or both eyes of the user in the visible light range of wavelengths. As described in further detail below, both the thermal image sensor 304 and the visible image sensor 306 (e.g., the data collected by these sensors) can be utilized to determine one or more dry-eye conditions associated with the eyes of the user.

In one or more examples, HMD 302 additionally includes one or more fans 308 that are configured, in part, to direct airflow toward the eyes of the user of the HMD 302. In one or more examples, the one or more fans 308 are disposed on HMD 302 such that at least a portion of the airflow generated by the one or more fans 308 impinges on the one or eyes of the user. As will be discussed in further detail below, the one or more fans 308 can be used by HMD 302 to mitigate a dry-eye condition based on a dry-eye condition that is determined using data from the thermal image sensor 304 and visible image sensor 306.

In one or more examples, and as described in further detail below, image and thermal data extracted from the eye (using the components described above with respect to FIG. 3) may be used for various purposes related to determining dry-eye conditions. As described in detail below, dry-eye conditions refer to conditions of the eyes of the user indicating that the eyes are not receiving a sufficient amount of moisture for various reasons (e.g., due to lack of blinking, and/or prolonged use of an electronic display). In one or more examples, the systems and methods described below can utilize sensor data, and various processing techniques to extract features from the sensor data, to determine dry-eye conditions. In one or more examples, extracting properties of one or both eyes of the user includes segmenting the sensor data to identify one or more regions of one or both eyes of the user. In one or more examples, and as described in further detail below, in response to determining a dry-eye condition, the electronic device (e.g., HMD 302) can provide (e.g., initiate or suggest) one or more mitigations that are configured to partially or fully mitigate the dry-eye condition.

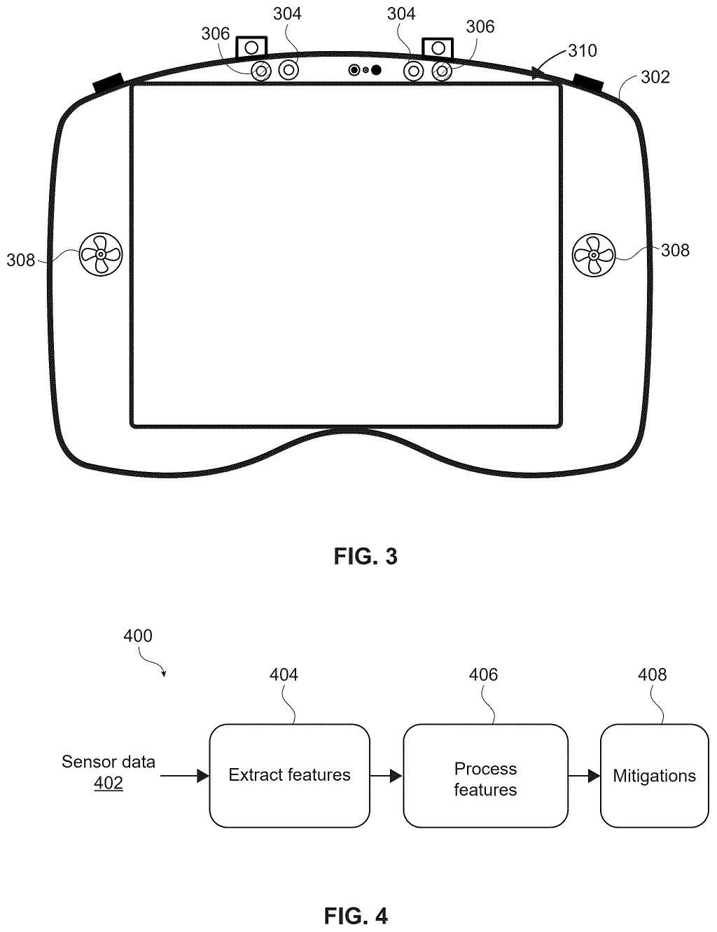

FIG. 4 illustrates an example dry-eye determination process for determining dry-eye conditions and providing mitigations according to one or more examples of the disclosure. In one or more examples, the process 400 of FIG. 4 accepts sensor data 402 as input (e.g., from the input data collected by thermal image sensor 304 and/or visible image sensor 306). As described above, the data collected by thermal image sensor 304 and visible image sensor 306 includes image data from one or more eyes of the user. In one or more examples, after obtaining the sensor data at 402, one or more features are extracted, at operation 404, from the data including (but not limited to) measurements of the eye, and/or other features associated with the eyes of the user as described in further detail below. In one or more examples, the one or more features/measurements are features used to determine a dry-eye condition.

In one or more examples, at operation 406, a dry-eye level/condition is determined from the extracted features (e.g., extracted at operation 404). In one or more examples, and as described in further detail, a dry-eye condition is determined when one or more criteria are satisfied. In one or more examples, the one or more criteria include at least one criterion that is based on the one or more extracted features. In one or more examples, the one or more criteria include multiple criteria based on the one or more extracted features. In one or more examples, when a dry-eye condition has been determined at operation 406, at operation 408, the electronic device (e.g., HMD 302) provides one or more mitigations for mitigating the dry-eye condition (described in further detail below).

The process 400 described above with respect to FIG. 4 can include various methods for determining a dry-eye condition that include extracting features (e.g., operation 404) from the image sensor data relating to the temperature of the eye, various spatial features of the eye, and anatomical features of the eye that can be used to determine a dry-eye condition. In one or more examples, the extracted features and measurements are used to determine dry-eye level, and subsequently mitigate dry-eye when appropriate.

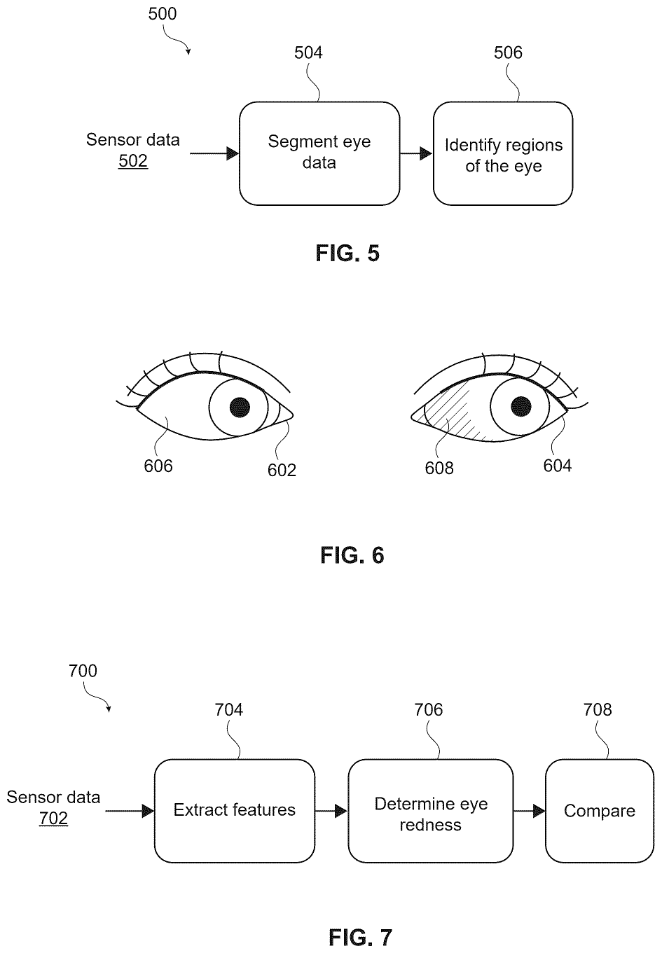

In one or more examples, as part of operation 404 of extracting features from the image sensor data, the electronic device can segment the image sensor data to identify various anatomical and spatial features of the eye as described below with respect to FIG. 5. As an example, the segmentation process can include identifying a tear film of the eye, blood vessels of the eye, and/or other anatomical features that can be further analyzed for indications of a dry-eye condition from the image sensor data.

FIG. 5 illustrates an example process as part of an extraction operation (e.g., operation 404) for segmenting image sensor data of the eyes of the user of electronic device to identify anatomical features of the eye according to examples of the disclosure. In one or more examples, at least part of the process 500 of FIG. 5 can be included as part of operation 404, in which various features are extracted from the image sensor data. In one or more examples, the process of FIG. 5 may be used to identify different parts of the eye such as the sclera, blood vessels, iris, tear film, pupil, and more, as described in further detail below. By identifying various regions of the eye through segmentation, the segmented regions may be used to determine dry-eye in the process described with respect to FIG. 4. In one or more examples, the segmentation process 500 of FIG. 5 begins by receiving the visible and thermal image sensor data 502 (e.g., corresponding to sensor data 402). At operation 504, the electronic device performs a segmentation process on the image sensor data to identify various anatomical features/regions of the eye, such as the pupil, the iris, various blood vessels, and other features that may be pertinent to determining a dry-eye condition. In one or more examples, the segmentation process includes applying a machine learning model to the received image sensor data, applying a semantic segmentation algorithm, applying an instance segmentation algorithm, and/or panoptic segmentation algorithm. In one or more examples, once the segmentation process has been applied at operation 504, at operation 506, the electronic device identifies one or more regions of one or more of the eye based on the outputs of the segmentation process. As described in further detail below, the anatomical features that are segmented in the process of FIG. 5 can be further analyzed to determine a dry-eye condition.

In one or more examples, the anatomical features that are segmented from the thermal and/or visible image sensor data includes the sclera of the eye (e.g., the white portion of the eye that covers most of the eyeball). In some examples, the color of the sclera can be indicative of a dry-eye condition. For example, the redness or redness intensity of the eyes may be indicative of a dry-eye condition. As described herein, redness or redness intensity may have different thresholds. For example, redness thresholds may include, but are not limited to: the total percent and/or amount of red of the sclera, number of regions of red (where multiple discreet regions could indicate a higher likelihood of dry eye, the shade of red (darker red indicating a potential higher intensity of dry eye, and any combination thereof. In some examples, the redness or redness intensity may be quantified by determining an average, minimum, maximum, mode, and median redness intensity (e.g., by processing red wavelength pixel density). For instance, in one or more examples, and as illustrated in FIG. 6, the sclera of the eye, and specifically the color of the sclera can be indicative of a dry-eye condition. For example, eyes may be redder due to dilated blood vessels or vasodilation caused by dryness on the surface of the eye. For instance, as illustrated in FIG. 6, the sclera 606 of eye 602 has a redness level that is below a threshold redness intensity (e.g., the amount of red in the sclera) of sclera 608 of eye 602 indicating that eye 604 is more likely to have a dry-eye condition (e.g., the moisture of eye 604 is not adequate).

In one or more examples, and as illustrated in FIG. 7, redness in the eye can be a feature extracted from the image sensor data and specifically visible image sensor 306 (described above with respect to FIG. 3). In one or more examples, process 700 uses sensor data 702, which optionally includes the image data from both the visible and thermal imaging sensors. In one or more examples, the sensor data 702 includes sensor data 402, 502 and/or information from process 400 and/or process 500 (e.g., segmented eye data, regions of the eye, etc.). In one or more examples, at operation 704, the electronic device extracts spatial properties and anatomical features of the of the one or both eyes of the user, including the sclera, from sensor data 702. In one or more examples, extracting spatial properties of one or both eyes of the user includes segmenting the sensor data to identify the one or more regions of one or both eyes of the user. In one or more examples, the electronic device identifies the sclera of the eye using the segmentation process described above with respect to FIG. 5. In one or more examples, after identifying the sclera of the eye of the user at operation 704 as part of the extraction, at operation 706, the electronic device determines a redness level of the eye. In one or more examples, the redness or redness intensity determined at operation 706 may be quantified by the red wavelength pixel density of the extracted images from the visible image sensor 306. For example, the redness or redness intensity may be quantified by determining an average, minimum, maximum, mode, and median redness intensity (e.g., by processing red wavelength pixel density). In some examples, specific areas of the eye are segmented based on redness or redness intensity. In some examples, at operation 708, the electronic device compares the redness level determined at operation 706 against a threshold (e.g., pre-determined threshold based on empirical study or based on a baseline for the user of the electronic device) to determine whether a dry-eye condition is indicated. In one or more examples, when the redness intensity is below the redness threshold, the eye is determined as a normal (e.g., pre-determined threshold based on empirical study or based on a baseline for the user of the electronic device) eye, or as not exhibiting a dry-eye condition. In contrast, when the redness intensity is at or above the redness threshold, the eye is determined as exhibiting a dry-eye condition.

Additionally or alternatively, in one or more examples, and as illustrated in FIG. 8, a method of determining eye redness may include determining the change in the diameter or thickness of the blood vessels of the eye. FIG. 8 illustrates two eyes including a first eye 802 and a second eye 804. In some examples, a “normal” (e.g., pre-determined threshold based on empirical study or based on a baseline for the user of the electronic device) eye (e.g., an eye without a dry-eye condition), represented by first eye 802, may exhibit “normal” (e.g., pre-determined threshold based on empirical study or based on a baseline for the user of the electronic device) permeability and blood flow, whereas a “red” eye, represented by second eye 804, may exhibit increased permeability and blood flow relative to first eye 802. Increased permeability and blood flow may be an indication of vasodilation, which may be caused due to a dry-eye condition. As illustrated in FIG. 8, non-vasodilated eye blood vessels 806 of first eye 802 may exhibit a “normal” (e.g., pre-determined threshold based on empirical study or based on a baseline for the user of the electronic device) diameter, whereas the vasodilated eye blood vessels 808 of second eye 804 may exhibit an increase in diameter during a dry-eye condition relative to first eye 802. The increase in diameter is illustrated by increased line width of the vasodilated blood vessels 808 compared with non-vasodilated eye blood vessels 806. The area between the blood vessels in FIG. 8 represent part of the scleral surface of the eyes, with scleral surface 810 of first eye 802 differentiated from the red scleral surface 812 of the second eye 804 exhibiting increased permeability and blood flow.

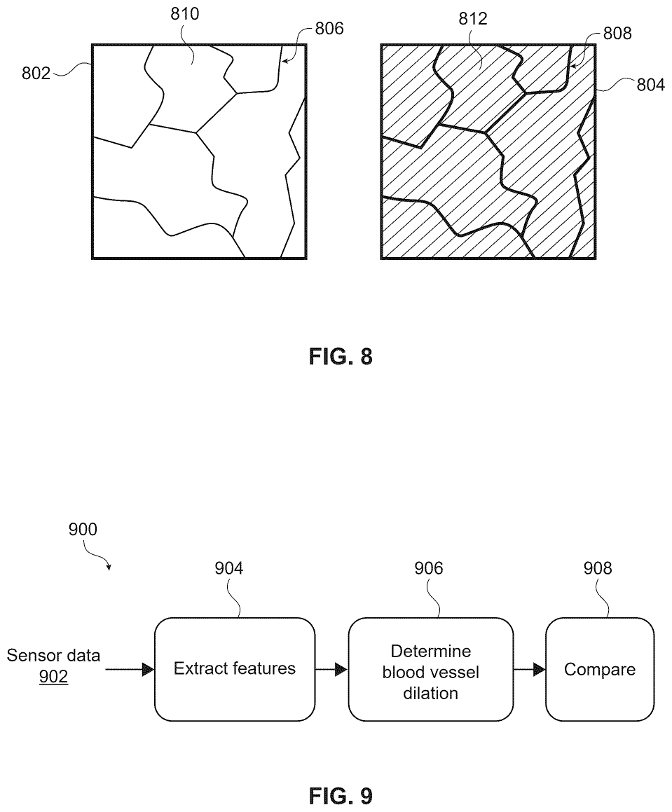

In some examples, and as illustrated in FIG. 9, blood vessel dilation of the eye can be a feature extracted from the image sensor data and specifically visible image sensor 306 (described above with respect to FIG. 3). In one or more examples, process 900 uses sensor data 902, which optionally includes the image data from both the visible and thermal imaging sensors. In one or more examples, the sensor data 902 includes sensor data 402, 502, 702 and/or information from processes 500 or 700 (e.g., segmented eye data, regions of the eye, redness levels, etc.). In one or more examples, at operation 904, the electronic device extracts properties of one or both eyes of the user to identify the sclera of the eye as part of the extraction. In one or more examples, the electronic device identifies the sclera of the eye using the segmentation process described above with respect to FIG. 5. In one or more examples, after identifying the sclera at operation 904 as part of the extraction, at operation 906, the electronic device determines a blood vessel dilation value. In one or more examples, the blood vessel dilation value determined at operation 906 may be quantified by the number of pixels in width of a blood vessel identified in an image of the eye, and specifically in the sclera of the eye extracted from images from the visible image sensor 306. In one or more examples, the blood vessel dilation value is determined for one blood vessel. In one or more examples, the blood vessel dilation value is determined for multiple blood vessels. In one or more examples, the blood vessel dilation value is determined based on statistic parameters for multiple blood vessels (e.g., mean, median, mode, maximum, minimum, variance, etc.). In some examples, after blood vessel dilation value is determined at operation 906, at operation 908, the electronic device compares the determined blood vessel dilation value against a threshold (e.g., pre-determined threshold based on empirical study or based on a baseline for the user of the electronic device) to determine whether a dry-eye condition is indicated. In one or more examples, when the blood vessel dilation value is below the threshold, the eye is determined as a normal (e.g., pre-determined threshold based on empirical study or based on a baseline for the user of the electronic device) eye, or as not exhibiting a dry-eye condition. In contrast, at 908 when the blood vessel dilation is above the threshold, the eye is determined as exhibiting a dry-eye condition.

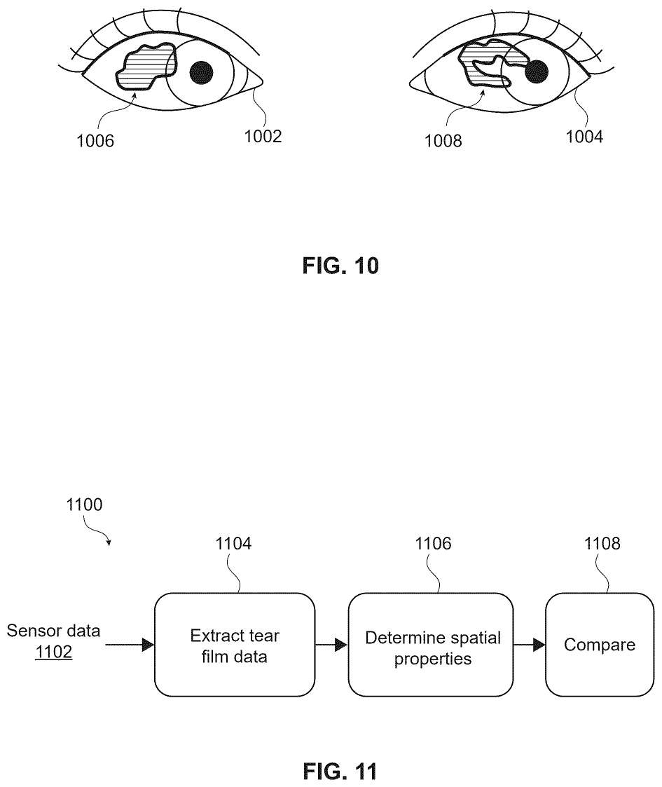

In some examples, the features extracted at operation 404 of FIG. 4 can include extracting spatial features of one or more tear film regions of the eyes of the user from the image sensor data acquired at process 400. In some examples, the features extracted at operation 404 of FIG. 4 may include extracting spatial features of one or more tear film thermal and/or temperature regions of the eyes of the user from the image sensor data acquired at process 400. In some examples, tear film thermal and/or temperature regions include thermal data of regions of the one or both eyes of the user. In one or more examples, spatial properties of the tear film regions of the eyes of the user can be used to determine whether the overall eye of the user has a dry-eye condition. FIG. 10 illustrates an example tear film region indicative of a dry-eye condition versus a tear film region indicative of a normal eye without a dry-eye condition according to one or more examples of the disclosure. FIG. 10 illustrates two eyes including a first eye 1002 and a second eye 1004. In the example of FIG. 10, first eye 1002 represents an image of a “normal” eye that is not indicating a dry-eye condition, whereas second eye 1004 represents an image of an eye that is indicating a dry-eye condition. In one or more examples, first eye 1002 includes tear film region 1006, whereas the second eye 1004 includes tear film region 1008. The tear film region 1006 and tear film region 1008 have different spatial features. The characterization of spatial features can be used to determine a dry-eye condition. In one or more examples, a measure of uniformity of the tear films is used to differentiate between a tear film corresponding to a dry-eye condition from a tear film corresponding to an absence of the dry-eye condition. For example, as depicted in FIG. 10, the tear film region 1006 has a relatively higher level of uniformity compared with tear film region 1008. The tear film region 1006 is relatively more uniform shape (e.g., roughly circular) indicative of a normal eye without a dry-eye condition, compared with tear film region 1008 is relatively non-uniform (e.g., includes irregularities) which are indicative of a dry-eye condition. In one or more examples, the measure of uniformity can be based on the perimeter of the tear film region, the area of the tear film region, or both (e.g., a ratio of the perimeter of the tear film region to the area of the tear film region). One or more thresholds can be applied to the one or more measures of uniformity to differentiate between an eye exhibiting the dry-eye condition and an eye is not exhibiting the dry-eye condition.

FIG. 11 illustrates an example process for determining a dry-eye condition based on spatial properties of the tear film region according to one or more examples of the disclosure. In one or more examples, at least part of process 1100 of FIG. 11 uses sensor data 1102, which optionally includes the image data from both the visible and thermal imaging sensors. In one or more examples, the sensor data 1102 includes sensor data 402, 502, 702, 902 and/or information from processes 500, 700 or 900 (e.g., segmented eye data, regions of the eye, redness levels, vasodilation level, etc.). In one or more examples, at operation 1104, the electronic device extracts properties from sensor data 1102, which includes segmenting the sensor data 1102 to identify the tear film region. In one or more examples, the tear film region is segmented from the image sensor data using the process 500 described with respect to FIG. 5. In one or more examples, the tear film region is identified or segmented based on temperature. In one or more examples, and as described in further detail below, the tear film region is identified by evaluating a cooling rate, wherein the cooling rate may be the decrease in temperature of one or more regions of one or both eyes over a period of time (e.g., a second, a minute, etc.). For example, one or more regions of the eye may be segmented based on the temperature of the one or more regions (e.g., within a threshold margin of variance in cooling rate). For example, one or more regions of the eye may be segmented based on the temperature (e.g., within a threshold margin of variance in temperature). In one or more examples, a cooling rate is determined for each temperature region. Due to the concentration of moisture allowing for faster evaporation, a tear film region may have a higher cooling rate compared to other regions of the eye. In one or more examples, the tear film region may be segmented as a higher cooling rate area compared to other areas of the surface of one or both eyes of the user of the electronic device. In one or more examples, the threshold variance for cooling rate differences varies based on factors such as time, environment, and features specific to one or both eyes of the user. In one or more examples, at operation 1106, the electronic device extracts one or more spatial properties from the tear film region. Extracted properties of the tear film region optionally include temperature, size, shape, moisture, color, and more. In one or more examples, the spatial properties that are determined at operation 1106 include properties that are indicative of an irregularly shaped tear film region. In some examples, the shape of the tear film regions includes the shape of segmented thermal regions on the tear film. In one or more examples, the ratio of the perimeter of the tear film region to the area of the tear film region can be determined at operation 1106. A large perimeter-to-area ratio can be indicative of a dry-eye condition. In one or more examples, at operation 1108, the electronic device compares the one or more spatial properties against one or more threshold values to determine whether a dry-eye condition is indicated. For example, at operation 1108, the perimeter-to-area ratio determined at operation 1106 is compared against a threshold (e.g., pre-determined threshold based on empirical study or based on a baseline for the user of the electronic device) to determine whether a dry-eye condition is indicated. In one or more examples, when the perimeter-to-area ratio value is below the threshold, the eye is determined as a normal eye, or as not exhibiting a dry-eye condition. In contrast, when the perimeter-to-area ratio is above the threshold, the eye is determined as exhibiting a dry-eye condition.

In one or more examples, the temperature of the eye, and specifically the rate at which the temperature decreases after a blink, can be indicative of a dry-eye condition. Thus, in one or more examples, and as describe below, the one or more features extracted at operation 404 of process 400 described above with respect to FIG. 4 can include temperature information about one or both eyes of the user.

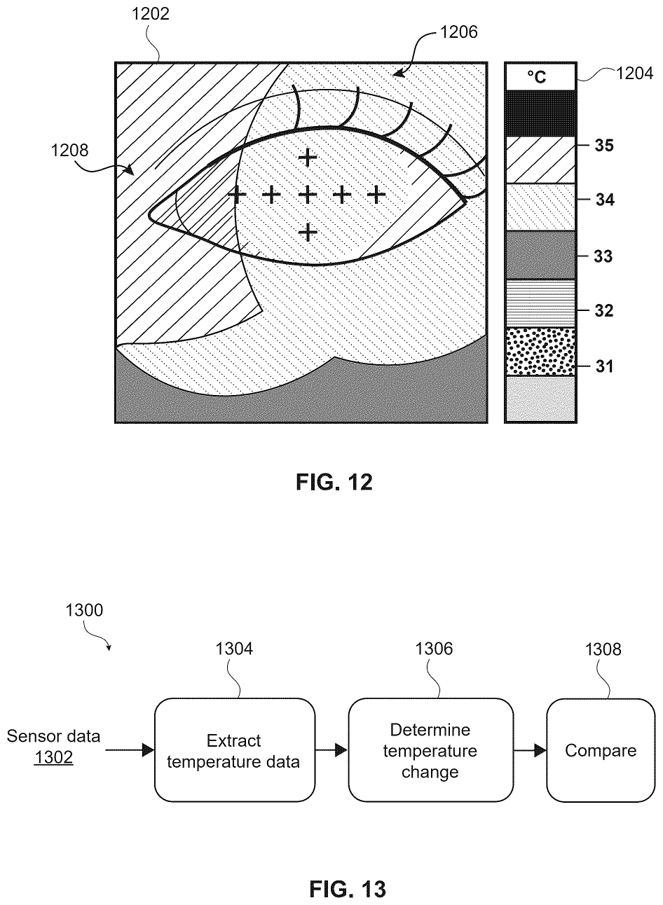

FIG. 12 illustrates an example heatmap of the various temperature regions of interest of the eye of the user according to one or more examples of the disclosure. In one or more examples, the image 1202 of the eye is captured by the thermal sensor 304 described above with respect to FIG. 3. In one or more examples, the temperature legend 1204 associated with image 1202 illustrates one or more temperature regions. In one or more examples, one or more temperature regions, such as temperature region 1206 and/or temperature region 1208 can be used to determine a cooling rate of the eye as described below with respect to FIG. 13. In one or more examples, temperature regions are identified using the one or more machine learning models that determine the temperature of regions such as of corresponding to tear film regions 1006 and 1008 shown in FIG. 10 corresponding to image 1202 (e.g., and specifically the estimated temperatures of those portions of the eye).

FIG. 13 illustrates an example process for determining a dry-eye condition using temperature of the eye according to one or more examples of the disclosure. In one or more examples, process 1300 uses sensor data 1302, which optionally includes the image data from both the visible and/or thermal imaging sensors. In one or more examples, the sensor data 1302 includes sensor data 402, 502, 702, 902, 1102 and/or information from processes 500, 700, 900 or 1100 (e.g., segmented eye data, regions of the eye, redness levels, vasodilation level, spatial properties, etc.). In one or more examples, the sensor data 1302 includes a plurality of images of the eye captured over a period of time that includes one or more images corresponding to a pre-determined amount of time after the eye has been determined to have blinked. In one or more examples, at operation 1304, the electronic device extracts the temperature data (e.g., the temperature of a portion of the eye at a given moment in time) from each image.

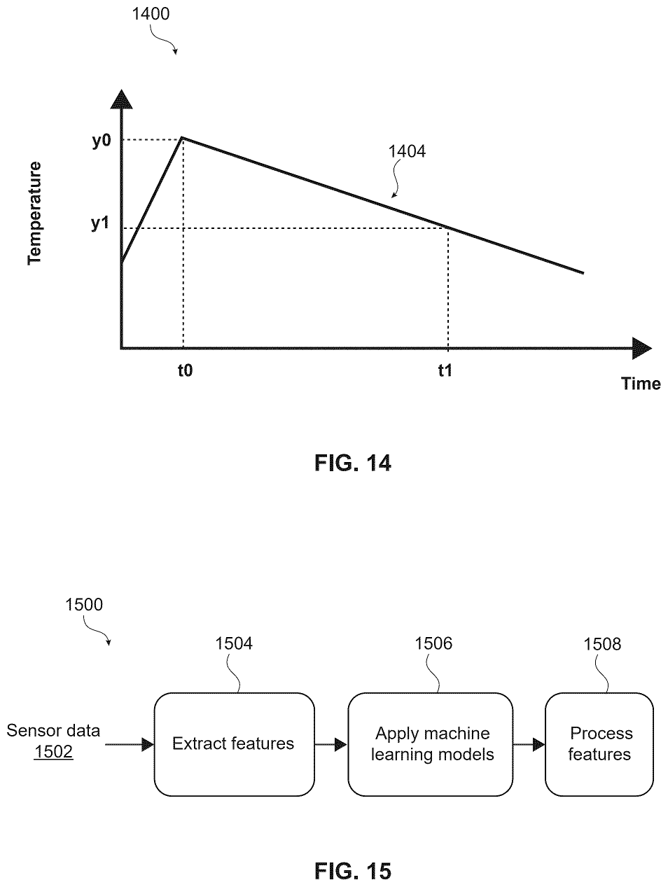

At operation 1306, the electronic device determines a temperature change of the one or both eyes of the user based on the temperature data of sensor data 1302. In one or more examples, a temperature change is a difference of extracted temperature. In one or more examples, a temperature change may be a difference of extracted temperature of various regions of the eye at various times. It is understood that a cooling rate can be calculated from the determined temperature change. FIG. 14 illustrates an example cooling rate 1404 according to one or more examples of the disclosure. FIG. 14 illustrates an example graph 1400 of the temperature of the eye (plotted temperature vs. time) and the cooling rate 1404 of an eye represented by the slope of the temperature data. For example, a temperature of a region of interest of the eye, such as the tear film temperature region, can be extracted at 1304. In graph 1400, a blink occurs from t=0 to t0, and at to the user opens the eye. While the eye is closed during a blink, the temperature of the eye increases due to friction from the rubbing of the eyelids and/or because evaporation of the tear film is limited by the closing of the eyelid. In one or more examples, the increase in temperature is indicated during a blink, with a peak temperature y0 indicative of the increased temperature at t0 during a blink. In one or more examples, after opening the eye, the eye cools due to the resumption of evaporation of the tear film. For example, at t1, the temperature of tear film region 1006 has cooled to temperature y1. In one or more examples, a cooling rate 1404 of the eye may be calculated with using the following equation:

$$cooling rate = ( y 0 - y 1 ) / ( t 1 - t 0 )$$

In one or more examples, the cooling rate 1404 may be calculated from the time when a peak temperature occurs (e.g., y0 at t1) until a pre-determined time has passed from the peak (e.g., at t1, a pre-determined time from t0).

Returning to the example of FIG. 13, after determining the cooling rate, at operation 1308, the electronic device compares the cooling rate against a threshold cooling rate to determine whether a dry-eye condition is indicated. For instance, when the cooling rate is below a threshold (e.g., pre-determined threshold based on empirical study or based on a baseline for the user of the electronic device), a dry-eye condition can be indicated (e.g., determined). When the cooling rate is above the threshold, the eye is determined as a normal eye, or as not exhibiting a dry-eye condition.

Extracting temperature data may be used in determining the cooling rate and consistency of the temperature at various regions of the eye. For example, the eye of the user may sharply increase in temperature after a blink due to the friction generated from the rubbing of the eyelids. As detailed further below, when the eyes cool at slower than a threshold rate, the temperature data may indicate reduced moisture and a slowness in evaporation, which may indicate a dry-eye condition.

In one or more examples, a machine learning model can be used to determine a dry-eye condition. For example, a machine learning model is trained (using a supervised or non-supervised training process) to determine a dry-eye condition directly from the images provided by the image sensors described above with respect to FIG. 3. In some examples, the machine learning model accepts images of one or both eyes from the image sensors. In some examples, the machine learning model output a representation of the eye condition. For example, the output can be a first state (“normal” or “non-dry-eye condition”) (e.g., pre-determined threshold based on empirical study or based on a baseline for the user of the electronic device) or a second state (“dry-eye condition”), or a probability that the eye exhibits a dry-eye condition (to which a threshold can be applied to determine a dry-eye condition or not). In some examples, the training includes a plurality of images and an indication (e.g., a label) of whether or not a dry-eye condition is exhibited.

FIG. 15 illustrates an example process for determining a dry-eye condition by applying one or more machine learning models to image sensor data according to examples of the disclosure. In one or more examples, process 1500 of FIG. 15 uses the sensor data 1502, which optionally includes the image data from both the visible and thermal imaging sensors. In one or more examples, the sensor data 1502 includes sensor data 402, 502, 702, 902, 1102, 1302 and/or information from processes 500, 700, 900, 1100 or 1300 (e.g., segmented eye data, regions of the eye, redness levels, vasodilation level, spatial properties, cooling rate, etc.). In some examples, sensor data 1502 are images without the additional information extracted from the images. At operation 1504, optionally one or more features of the eye are extracted. In some examples, one or more features of the eye are not extracted prior to the application of the machine learning model at operation 1506. In one or more examples, at operation 1506, the electronic device applies one or more machine learning models that are configured to determine a probability that the image includes an eye that indicates a dry-eye condition. In one or more examples, the output generated by the machine learning model at operation 1506 is processed to determine a dry-eye condition at operation 1508. For example, at operation 1508, the output of the machine learning model at operation 1506 is compared against a threshold (e.g., pre-determined threshold based on empirical study or based on a baseline for the user of the electronic device) to determine whether a dry-eye condition is indicated. In one or more examples, when the output of the machine learning model is below the threshold, the eye is determined as a normal eye, or as not exhibiting a dry-eye condition. In contrast, when the output of the machine learning model is at or above the threshold, the eye is determined as exhibiting a dry-eye condition.

In some examples, the optional extraction of features at 1504, or the extraction of features of the aforementioned processes (e.g., at 404, 704, 904, 1106, 1304, 1504) can be implemented by one or more machine learning models. For example, respective machine learning models can be trained to accept sensor data as inputs and to output respective features (e.g., based on training data including raw images and extracted features/feature-labeled images). For example, a respective machine learning model outputs an indication of segmented eye data, regions of the eye, redness levels, vasodilation levels, spatial properties, cooling rate, etc.

Returning to the example of FIG. 4, once a dry-eye condition has been determined at operation 406, the process 400 of FIG. 4 can include, at operation 406, the electronic device providing one or more mitigations to mitigate the dry-eye condition. In one or more examples, the mitigation includes prompting a user to take a break from use of the electronic device or apply moisture to the eyes (e.g., via warm compress or drops), and/or initiating operation of fans, among other mitigations. The mitigations allow the eyes to rehydrate and return to baseline metrics. In one or more examples, when one or more criteria for determining dry-eye are satisfied at operation 406, the HMD 302 notifies the user about the dry-eye condition and/or mitigations for a dry-eye condition. For example, the notification optionally includes visual, audio, and/haptic feedback. For example, the HMD 302 optionally displays a visual indication to encourage the user to pause use of the device and/or wear the device in a manner that may mitigate the dry-eye condition. For example, the HMD 302 optionally displays a visual indication about initiating fans or options to rehydrate the eyes.

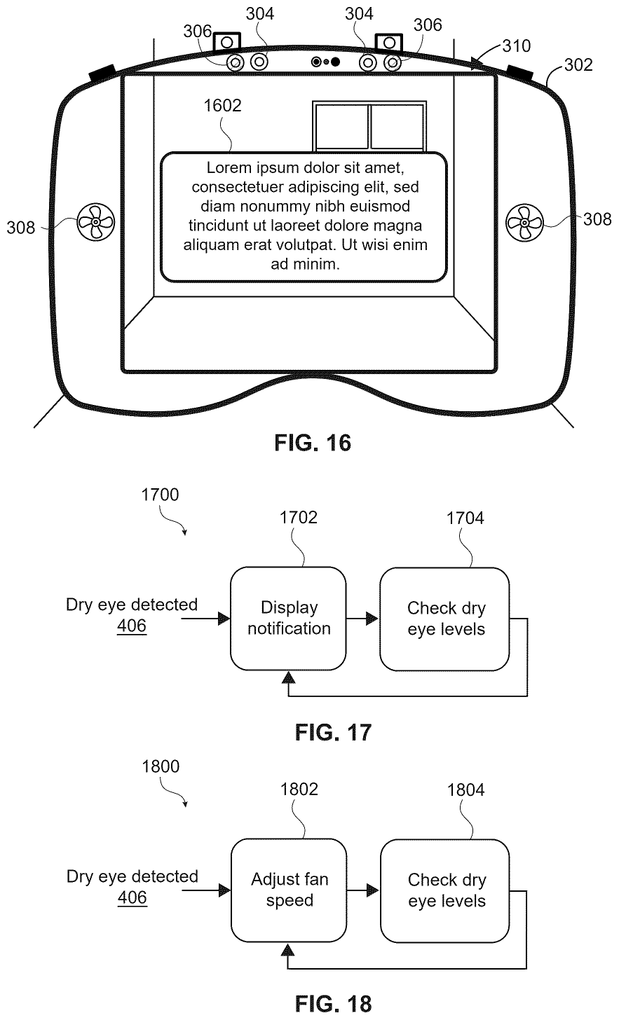

FIG. 16 illustrates an example visual notification displayed by the electronic device to instruct the user on a mitigation for improving a dry-eye condition according to examples of the disclosure. In one or more examples, the electronic device displays notification 1602 in response to determining a dry-eye condition. For instance, as an example, the electronic device may display messages such as “Remember to take a break occasionally while wearing the HMD to rest your eyes. If you are feeling symptoms of dry-eye, you can apply a warm compress to your eyes, use tear drops, or rest your eyes.” In one or more examples, the visual notification 1602 is accompanied by a notification sound (e.g., from speakers 216), haptic feedback (e.g., from a haptic generator, not shown), or any combination thereof that is configured to emphasize the visual notification 1602. In one or more examples, the visual notification 1602 is displayed on the display 310 of the HMD 302 periodically as dry-eye levels are continuously monitored. In one or more examples, the visual notification 1602 includes a selectable button which gives the user an option to close or mute the notification 1602. In one or more examples, the electronic device in addition to providing visual notification 1602 may prevent further use of the HMD 302 without determining a reduction in current dry-eye levels. In one or more examples, the visual notification 1602 may provide relevant info such as the user's eye metrics described above (e.g., cooling rate 1404), as well as provide historical data on the user's previously determined dry-eye incidents. In some examples, warning the user includes a notification 1602 which includes: providing a notification based on the information, sending a message based on the information, displaying the information, controlling a user interface of an application based on the information, controlling a user interface of a health application and logging eye related health data of the user to the health application based on the information, controlling a focus mode based on the information, setting a reminder based on the information, or adding a calendar entry based on the information, or any combination thereof.

In one or more examples, the process 1700 of FIG. 17 includes, after a dry-eye is determined at operation 406, operation 708, operation 908, operation 1108, operation 1308, operation 1508, operation 1804, or any combination thereof, displaying a notification (such as notification 1602 described above) at operation 1702. In one or more examples, at operation 1704 the electronic device checks the dry-eye condition of the user (according to one or more of the processes described above) to determine if the dry-eye condition has been mitigated or otherwise resolved. In one or more examples, at 1704, the dry-eye levels are rechecked after a certain period of time (e.g., 1 minute, 5 minutes, etc.). When the dry-eye condition continues to be determined, the notification 1602 is redisplayed (or continues to be displayed if not dismissed). In one or more examples, the electronic device, in response to determining a threshold (e.g., 5 dry-eye conditions) of dry-eye conditions over a threshold period of time (e.g., a minute, an hour, etc.), displays notification 1602 based on the dry-eye condition. The electronic device can set a threshold number of dry-eye determinations before applying mitigations so as to reduce a possibility of false positive dry-eye determinations, (e.g., where the system determines a dry-eye condition when one is not present).

In one or more examples, additionally or alternatively to displaying a notification 1602, the electronic device can modify the blinking behavior of the user, including inducing the user to blink by adjusting and/or modulating the speed of one or more fans of the device that provide air flow to eyes of the user.

FIG. 18 illustrates an example process for adjusting the speed of the fan to mitigate a dry-eye condition according to one or more examples of the disclosure. In one or more examples, the process 1800 of FIG. 18 can adjust the speeds of fans 308 of the HMD 302 in response to detecting a dry-eye condition at 1802. In one or more examples, when one or more criteria are satisfied indicative of a dry-eye condition, the one or more fans 308 are activated and/or the speeds of one or more fans 308 of the HMD 302 may be increased to direct more airflow at operation 1802 toward one or both eyes of the user. Directing increased airflow at operation 1802 to the eyes may modify blinking behavior of the user, including inducing the user to increase blink frequency, thereby mitigating or reducing dry-eye levels as tear production may increase due to the increased blinking frequency. In one or more examples, fan speeds of the one or more fans 308 are lowered to prevent increased dry-eye levels. For example, when increasing the fan speeds at operation 1802 does not increase blink frequency, the fan speeds may be lowered below a predetermined threshold value to prevent increased dry-eye levels by lowering the cooling rate due to reduced evaporation of tears caused by the reduced airflow. In some examples, after the fan speed has been adjusted at 1802, at 1804 the electronic device checks if the dry-eye condition is still present and if so, can adjust the fan further.

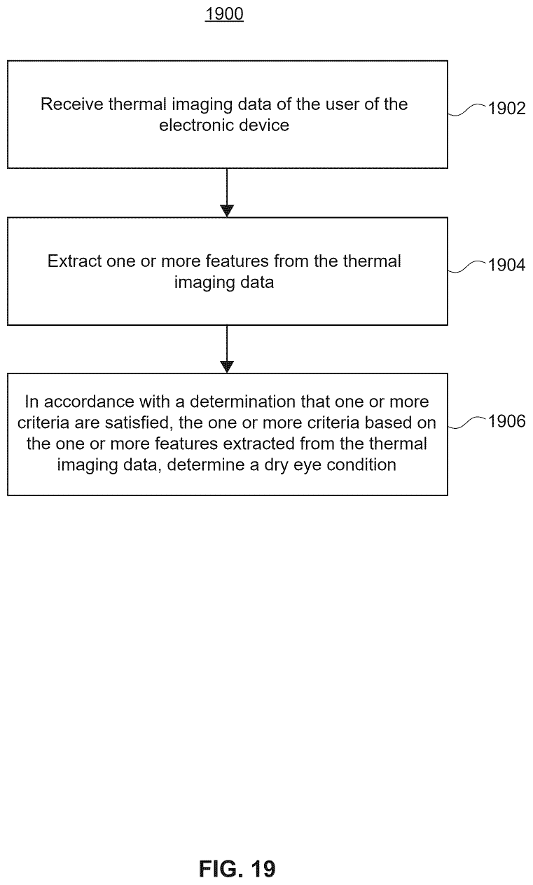

FIG. 19 illustrates an example method for determining a dry-eye condition according to examples of the disclosure. In one or more examples, the process 1900 is performed at an electronic device in communication with one or more displays and/or one or more input devices including one or more thermal image sensors configured to capture thermal imaging data of one or both eyes of a user of the electronic device. For example, the electronic device is a mobile device (e.g., a head mounted display, smart eyeglasses, a tablet, a smartphone, a media player, or a wearable device) including wireless communication circuitry, optionally in communication with one or more of a mouse (e.g., external), trackpad (optionally integrated or external), touchpad (optionally integrated or external), remote control device (e.g., external), another mobile device (e.g., separate from the electronic device), a handheld device (e.g., external), and/or a controller (e.g., external), etc. In one or more examples, the display generation component is a display integrated with the electronic device (optionally a touch screen display), external display such as a monitor, projector, television, or a hardware component (optionally integrated or external) for projecting a user interface or causing a user interface to be visible to one or more users, etc. In one or more examples, the electronic device is part of an electronic device that is part of a wearable device. Examples of input devices include an image sensor (e.g., a camera), thermal sensor, spectrophotometer, location sensor, hand tracking sensor, eye-tracking sensor, motion sensor (e.g., hand motion sensor) orientation sensor, microphone (and/or other audio sensors), touch screen (optionally integrated or external), remote control device (e.g., external), another mobile device (e.g., separate from the electronic device), a handheld device (e.g., external), and/or a controller.

In one or more examples, the electronic device receives, at 1902, the imaging data of the user of the electronic device, including thermal imaging data. In some examples, the one or more thermal image sensors can include Indium Gallium Arsenide (“InGaAs”) photodetectors or any other type of thermal imaging sensors, such as a passive or an active infrared (IR) sensor, for detecting IR light (e.g., visible light sensor, IR sensor, etc.) including infrared ocular thermal imaging sensors. For example, an active IR sensor includes an IR emitter for emitting infrared light into the real-world environment. In one or more examples, the thermal image sensors can include a visible light camera that detects visible light reflecting off the eyes of the user. The thermal image sensors may be placed such that the sensors have a clear and unobstructed view of one or both eyes of the user thus allowing the sensors to be used to take measurements of one or both eyes during operation of the electronic device. The thermal image sensors may record video and take photos as directed by the device (e.g., the electronic device is communicatively coupled to the thermal image sensor and is configured to send commands to the image sensor to take photos and/or video). In some examples, the thermal image sensors may record data on the infrared energy, or heat signature, of one or both eyes of the user of the electronic device, including an electronic image. In some examples, the image sensors optionally include one or more visible light image sensors, such as charged coupled device (CCD) sensors, and/or complementary metal-oxide-semiconductor (CMOS) sensors operable to obtain images of the eye(s) of the user of the electronic device. Image sensors also optionally include one or more cameras configured to capture movement of physical objects in the real-world environment. Image sensors also optionally include one or more depth sensors configured to detect the distance of physical objects from electronic device. In some examples, information from one or more depth sensors can allow the device to sense and differentiate objects in the real-world environment from other objects in the real-world environment. In some examples, one or more depth sensors can allow the device to determine the texture and/or topography of objects in the real-world environment. In some examples, the data collected from the thermal image sensor(s) may be combined with the data collected from the visible image sensor(s) to produce electronic images blending the temperature and image data. In some examples, the blended temperature and image data includes a color map of the apparent temperature of the eye(s) of the user of the electronic device.

In one or more examples, the electronic device extracts, at 1904, one or more features from the thermal imaging data. In some examples, the extracted features are stored on the electronic device and/or a storage device in communication with the electronic device. In some examples, the storage device is a cloud storage device including a database of related content. In some examples, the extracted features may include various physical properties of the eye(s) of the user. The physical properties that are extracted can include but are not limited to temperature, moisture, size, shape, color (including ultraviolet, visible, and infrared light), texture, and/or luster. In some examples, the one or more features include patterns extracted from the thermal imaging data, including threshold values.

In one or more examples, in accordance with a determination that one or more criteria are satisfied, the one or more criteria based on the one or more features extracted from the thermal imaging data, the electronic device determines, at 1906, a dry-eye condition. In one or more examples, the electronic device, in response to determining a threshold number of determined dry-eye conditions have been met over a threshold period of time (e.g., a minute, an hour, etc.), provides one or more mitigations based on the dry-eye condition. In an example, the threshold number of determined dry-eye conditions is 5 determinations, 4 determinations, less than 4 determinations, or greater than 4 determinations. In one or more examples, setting threshold number of dry-eye determinations before applying mitigations may reduce a possibility of false positive dry-eye determinations, where the system determines a dry-eye condition when one is not present. In one or more examples, dry-eye determinations are reduced to a threshold number when there is an increased rate of dry-eye determinations a threshold number of times (e.g., 5 dry-eye determinations) in a threshold period of time (e.g., a minute, an hour, etc.). In one or more examples, reducing dry-eye determinations after exceeding a threshold number may reduce computational strain on the system, and optionally reduce the drain on a batter if one is present in the system. Additionally, in one or more examples, in accordance with a determination that one or more criteria are satisfied, the electronic device provides one or more mitigation. In one or more examples, in accordance with a determination that one or more criteria are not satisfied, the electronic device forgoes determining a dry-eye condition.

In some examples, deep learning and/or machine learning method(s) are used to determine current dry-eye level based on the one or more extracted features. For example, the electronic device can include one or more machine learning algorithms such as neural networks (e.g., convolutional neural networks), supervised and/or unsupervised machine learning algorithms, and/or the like. The machine learning method(s) may use numerical, categorical, time-series, and/or text data. In one or more examples, the machine learning model(s) may be trained on the cloud, connected to the cloud during use, trained locally, and/or a combination thereof. In one or more examples, threshold factors of the extracted features are determined for determining a dry-eye condition. For example, in some examples, threshold factors can include but are not limited to blink frequency, blink completion, spatial properties of tear film temperature regions (e.g., shape, size, etc.), cooling rate, temperatures at various regions of interest, eye redness, blood vessel dilation, and more, or any combination thereof. The threshold values of the determined threshold factors can be predetermined based on the specific attributes of the user of the electronic device, including but not limited to: age, sex, race, ethnicity, location, user history, and other information, or any combination thereof.

In one or more examples, the one or more features extracted from the thermal imaging data include spatial properties of tear film temperature regions of one or both eyes of the user of the electronic device. In some examples, the tear film is a fluid that coats the surface of the eye, specifically the cornea and conjunctiva or sclera, and plays a role in maintaining eye health and vision. In some examples, the tear film protects the eye from environmental irritants, keeps the eye moist, and provides nutrients to the cells of the eye. Disruptions or deficiencies in the tear film stability may lead to a dry-eye condition or other ocular surface diseases. In some examples, the method involves extracting one or more features from thermal imaging data including extracting spatial properties of tear film temperature regions of the eyes of the user of the electronic device. The thermal imaging data may be captured using thermal sensors or cameras that extract variations in temperature across the surface of the eyes. In one or more examples, extracting variations in temperature of one or both eyes of the user include segmenting the data to identify the one or more regions of one or both eyes of the user. For example, the data is optionally processed to identify distinct regions of the tear film that exhibit varying temperature characteristics. In some examples, the spatial properties refer to geometric and positional attributes, such as the shape, size, location, and distribution patterns of the tear film temperature regions within the thermal image. These properties may be quantified through image processing techniques that segment and analyze the tear film based on identified temperature variations. In some examples, the analysis of spatial properties of tear film temperature regions is utilized for diagnostic or monitoring purposes. In some examples, the determined spatial properties are used to assess tear film stability.

In one or more examples, extracting spatial properties of tear film regions of one or both eyes of the user include segmenting the thermal imaging data to identify the one or more tear film temperature regions. In some examples, segmenting refers to the process of partitioning an image into multiple segments or regions, each representing a distinct object, feature, or area of interest within the image. In some examples, the goal of segmentation may be to simplify or change the representation of an image into something to analyze by isolating relevant parts from the rest of the image. In some examples, image processing techniques, including thermal gradient analysis, segmentation algorithms, and pattern recognition methods, are employed to extract spatial properties from the thermal imaging data. The processing steps may include filtering, image thresholding, edge detection, region growing methods, watershed segmentation, or feature extraction algorithms designed to identify and isolate relevant temperature regions within the tear film. These segmentation techniques may be used alone or in combination with each other or with additional processing steps to identify the one or more tear film temperature regions. In some examples, the spatial properties of the identified tear film regions may include, but are not limited to: temperature values, spatial distributions (e.g., heat maps), region sizes and shapes boundaries and contours. In some examples, the method may further comprise analyzing the segmented tear film regions to identify patterns or anomalies within the tear film region that indicate a potential dry-eye condition. This analysis may be performed using various techniques, including but not limited to: statistical processing, machine learning algorithms, and pattern recognition methods. The results of this analysis can be used to provide feedback to the user, such as alerts or warnings about potential dry-eye conditions, and/or to trigger additional processing steps, such as generating a report or sending data to a healthcare provider.

In one or more examples, the spatial properties include shape. The shape of the tear film regions can be extracted and processed to determine various characteristics, such as boundary irregularities, region convexity and concavity, shape asymmetry, size, and aspect ratio. In one or more examples, extracting various characteristics of one or both eyes of the user includes segmenting the data to identify the one or more regions of one or both eyes of the user. In some examples, segmenting the data includes segmenting the tear film to identify the spatial properties (e.g., shape) of the segmented thermal regions on the tear film. In some examples, the spatial properties include size of the segmented thermal regions on the tear film. In some examples, determining the size and/or shape of the tear film regions includes determining the size and/or shape of the segmented thermal regions on the tear film. For example, these shape-based properties can be used to identify potential dry-eye conditions by comparing them to known shapes associated with tear film regions that are within a threshold range of tear film regions associated with an average healthy human eye. In some examples, the method may further comprise analyzing the shape-based properties to determine changes in the tear film regions over time, such as changes in boundary and perimeter irregularities, shifts in convexity and concavity, or alterations in shape asymmetry. These changes can be indicative of a developing dry-eye condition, and the method may trigger alerts or warnings accordingly when determining changes in the shape of the tear film. In some examples, the shape of the tear film regions includes the shape of segmented thermal regions on the tear film. Additionally, these shape-based properties can be used to track the effectiveness of treatment for dry-eye conditions. The identified shapes can also be used to compare with normal and abnormal (e.g., pre-determined threshold based on empirical study or based on a baseline for the user of the electronic device) tear film region shapes in a database, allowing for identification of potential dry-eye conditions. The comparison can be performed using various techniques, including but not limited to: image processing algorithms, machine learning models, neural networks, or statistical analysis methods.

In one or more examples, the spatial properties include size. In some examples, the tear film region size and various characteristics are determined, such as: size variation between regions, regional growth or shrinkage over time, and relative sizes of adjacent regions. These size-based properties can be used to determine potential eye conditions by comparing them to known sizes associated with tear film regions that are within a threshold range of tear film regions associated with an average healthy human eye. For example, an abnormally large (e.g., larger than a pre-determined threshold size based on empirical study or based on a baseline for the user of the electronic device) or small region (e.g., smaller than a pre-determined threshold size based on empirical study or based on a baseline for the user of the electronic device) may indicate a developing eye condition. In some examples, the method may further comprise analyzing the size-based properties to determine changes in the tear film regions over time, such as: changes in regional growth or shrinkage rates (as the tear film may naturally grow or shrink over time) shifts in relative sizes of adjacent regions, or alterations in overall tear film volume. These changes can be indicative of a developing eye condition, and the method may trigger alerts or warnings accordingly. The identified size properties can also be used to compare with known within threshold and outside of threshold tear film region sizes stored in a database, allowing for identification of potential eye conditions. The comparison can be performed using various techniques, including but not limited to: image processing algorithms, machine learning models, neural networks, or statistical analysis methods.

In one or more examples, the one or more features extracted from the thermal imaging data include spatial properties of one or more temperature regions of the eye. In some examples, the method may extract imaging data from various regions of the eye (such as the tear film temperature region or area). In one or more examples, the regions are optionally determined through their temperature differences. In some examples, these spatial properties may include characteristics such as tear film area and perimeter, which can be calculated using various techniques, including but not limited to: image processing algorithms, neural networks, or statistical analysis methods. In some examples, machine learning methods such as edge detection may be used in conjunction with thresholding, morphological analysis, distance calculation (e.g., measuring the pixel distance between detected edges), or any other machine learning methods or combinations thereof to measure and calculate the area and perimeter of tear film area, or any combination thereof. The tear film area and perimeter can provide information about one or both eyes of the user, particularly in relation to dry-eye conditions. For example, a ratio of tear film area to perimeter that is outside a threshold range of a ratio tear film are to perimeter associated with an average healthy human eye may indicate a developing dry-eye condition. The method may compare the ratio to one or more threshold values, which can be determined by various methods. The threshold values may be adjusted based on various factors specific to the user, including but not limited to age, gender, sex, race, ethnicity, location, user history, and environmental conditions. In some examples, the threshold values are determined using statistical analysis, machine learning algorithms, image processing techniques, clinical trials, and more. In some examples, the ratio of the area to the perimeter of the extracted tear film region is used to determine dry-eye levels. This determination can include determining even or uneven cooling of the eye. In one or more examples, a high ratio of the area to the perimeter of the extracted tear film region can indicate dry-eye, whereas a lower tear film ratio can function as a baseline threshold value depending on the user of the electronic device.

In one or more examples, the one or more features extracted from the thermal imaging data include a cooling rate of one or both eyes of the user of the electronic device. In some examples, the cooling rate of one or both eyes of the user refers to the rate at which the temperature of the eye cools over time after a stimulus, such as a blink or a change in environmental conditions. Determining the cooling rate can provide insights into the health and function of the ocular surface of one or both eyes of the user of the electronic device. A cooling rate that is within a threshold range of cooling rates associated with an average healthy human eye cooling rate cooling rate may indicate healthy tear film function and adequate ocular surface lubrication or hydration. The tears help to regulate the temperature of the eye by dissipating heat away from the cornea and conjunctiva or sclera. When the eyes are in a state that is within a threshold range of states associated with an average healthy human eye (e.g., not in a dry-eye condition), the cooling rate may be above a threshold value that indicates rapid cooling of the eye, indicating that the tears are sufficiently removing heat from the eye. For example, an abnormal cooling rate (e.g., outside of a pre-determined threshold range based on empirical study or based on a baseline for the user of the electronic device) may indicate dry-eye or other eye conditions. For example, a slower than baseline threshold cooling rate may indicate a reduction in tear film quality or quantity, which can increase the risk of dry-eye development. This may happen because the tears are not able to effectively dissipate heat from the eye, leading to accumulation of heat and potential dryness and/or damage to the ocular surface. Measuring the cooling rate of one or both eyes of the user may be a method of determining the evaporation rate of the tears of the eyes. During a blink when the eyelids generate heat, the surface temperature of the eye may be warmest. Immediately after a blink, when the eyelids open, tears evaporate and cool the surface of the eye. In some examples, the cooling rate is determined by an average temperature measurement of one or more regions of interest of the eye over a period of time (e.g., millisecond, second, minute, etc.) or frames (e.g., temperature per frame). In some examples, the temperature measurement is of the lowest temperature region of one or both eyes of the user of the electronic device. In some examples, the temperature measurement is of the highest temperature region of one or both eyes of the user of the electronic device. In some examples, the cooling rate is determined by a combination of different temperature regions that may be weighed differently in determining the cooling rate over a certain period. Parts of the eye may have different cooling rates. In some examples, the thermal imaging data is segmented to determine cooling rate at various regions of the eye, including the lowest temperature region of the eye known as the tear film. The cooling rate of the tear film may give indications to the quality and stability of the tear film.

In one or more examples, the cooling rate is a change in temperature over a predetermined duration measured from a first time after a blink to a second time, after the first time, while one or both eyes remain open. In some examples, machine learning models are introduced in evaluating the blinking condition, enabling the method to learn patterns and relationships within the extracted features. In some examples, the method applies one or more machine learning models to the one or more features to evaluate the blinking condition. In some examples, the machine learning models can be trained using labeled datasets of features extracted from imaging data, where the labels represent the desired output or determination. This allows the method to learn patterns and relationships within the extracted features that are relevant to the task at hand. In some examples, by applying machine learning models in evaluating the blinking condition, the method can determine whether a blinking condition is present or not. In some examples, the predicted outcomes can be used in conjunction with other methods related to image processing, blink detection, and eye tracking to develop more models of eye function and predict potential issues related to dry-eye or other conditions. In some examples, the machine learning models play a role in evaluating the one or more criteria and determining a blinking condition. In some examples, analyzing the extracted features, these models can determine patterns and relationships, enabling them to predict whether a blinking condition is present or not. For example, a machine learning model trained on thermal imaging data may be able to detect changes in corneal temperature or heat flux that are indicative of dry-eye, even before symptoms become apparent. In some examples, a machine learning model analyzing spatial properties such as eyelid position and shape may be able to identify abnormal (e.g., pre-determined threshold based on empirical study or based on a baseline for the user of the electronic device) blinking patterns that indicate increased risk of dry-eye. In some examples, the method can also use transfer learning to leverage pre-trained models for classification or regression tasks, allowing for faster training times and improved performance. In some examples, the method can use ensemble methods to combine the predictions from multiple machine learning models to improve overall accuracy and robustness. The extracted features can include spatial properties such as eye size and shape, position and orientation, distance between the eyes, shape and size of the eyelids, shape and size of the pupils, iris texture and pattern, corneal curvature and shape, conjunctival folds and creases, orbital rim, facial structure, and more, or any combination thereof. In some examples, the method can also extract thermal imaging data, including temperature, heat flux, and thermal conductivity. In some examples, by combining the predictions from multiple models, the method can provide a comprehensive evaluation of the one or more criteria and determine whether a blinking condition is present or not, allowing for early intervention and prevention of dry-eye conditions. In some examples, the predetermined duration measured from a first time after a blink to a second time can be determined using various methods, including but not limited to: fixed time intervals, dynamic analysis of temperature changes, or adaptive learning algorithms. In some examples, the predetermined duration may be a fixed value, such as 1 millisecond, 30 milliseconds, 100 milliseconds, 1 second, or more, which is used to measure the cooling rate. This can provide a standardization measurement for comparing the cooling rates across different users and conditions. In some examples, the predetermined duration may be dynamically adjusted based on various factors, including but not limited to: the type of stimulus (e.g., blink or environmental change), ambient temperature, humidity, the user's age, gender, sex, race, ethnicity, geography, ocular health or other history. Measuring the cooling rate after a blink can provide insights into the health and function of the ocular surface. The blink is a natural reflex that helps to spread tears across the cornea and conjunctiva or sclera, providing lubrication and protection to the eyes. By measuring the cooling rate immediately following a blink, the method can capture the rapid changes in temperature that occur as the tears begin to evaporate. In some examples, to measure the cooling rate after a blink, the system may use various techniques, including but not limited to: thermal imaging, infrared spectroscopy, or thermocouple-based measurements. For example, the system may use a thermal camera to capture images of the eye before and after a blink, and then analyze the changes in temperature over time using image processing algorithms. In some examples, the method may also include additional steps to enhance the accuracy of the cooling measurement. For example, the system may use noise reduction techniques to minimize artifacts in the thermal imaging data, or apply filters to remove unwanted signals from the measurement.

In one or more examples, extracting one or more features from the thermal imaging data includes segmenting the thermal imaging data to identify a first region of one or both eyes in the thermal imaging data, and extracting a temperature of the first region from the thermal imaging data. In some examples, the first region may be a specific part of the eye, including but not limited to: the cornea, conjunctiva, sclera, iris, pupil, or retina. The first region may be identified using various techniques, including but not limited to: automated thresholding, or machine learning-based object detection algorithms. In some examples, the method may employ automated thresholding techniques to identify the first region based on predetermined temperature thresholds. For example, the system may set a temperature threshold for the cornea (e.g., 35 degrees Celsius) and use automated thresholding to identify areas of the eye that meet this threshold. In some examples, the method may use machine learning-based object detection algorithms to identify the first region. These algorithms can be trained on a dataset of thermal imaging data labeled with specific regions of interest (e.g., cornea, conjunctiva, sclera, iris) and then applied to new images to automatically identify those regions. The temperature of the identified region can be extracted using various techniques, including but not limited to: image processing algorithms, machine learning models, neural networks, or statistical analysis methods. The properties that are extracted can include but are not limited to temperature, moisture, size, shape, color (including ultraviolet, visible, and infrared light), texture, and/or luster. In some examples, the one or more features include patterns extracted from the thermal imaging data, including threshold values.

In one or more examples, determining a dry-eye condition comprises applying one or more supervised machine learning (ML) models (e.g., implemented as hardware or using hardware to implement software and/or firmware) to the one or more features extracted from thermal imaging data. In one or more examples, ML models determine one or more threshold values to determine a blinking condition. In some examples, the ML model is trained on one or more features and measurements from the eye in determining the threshold values. In one or more examples, ML models determine one or more criteria (e.g., blinking condition) to determine a dry-eye condition. In some examples, the ML model is trained on one or more features and measurements from the eye in determining the criteria. In one or more examples, a first ML model (e.g., a convolutional neural network model implemented as hardware or using hardware to implement software and/or firmware) determines a blinking condition (e.g., open or close state, complete, partially incomplete (right), partially incomplete (left), incomplete, and complete blinks) of one or both eyes of the user of the device. In one or more examples, the first ML model then determines satisfaction of one or more criteria and determines a dry-eye condition if satisfaction is achieved. In one or more examples, a threshold number of dry-eye determinations are required for application of dry-eye mitigations such as warning notifications to the user of the electronic device. The threshold number of dry-eye determinations may change dynamically depending on factors such as environment, time (e.g., of day, week, etc.), and factors specific to the user. In one or more examples, the extracted features are filtered and inputted to a second ML model (e.g., implemented as hardware or using hardware to implement software and/or firmware) model (e.g., a convolutional neural network) that is trained on the extracted features. In some examples, the output from the first ML model is inputted to the second ML model. In some examples, the second ML model may determine the satisfaction of one or more criteria such as the upper eyelid touching the lower eyelid of one or both eyes of a user of the electronic device in determining a blinking condition of the user. In one or more examples, the second ML model then determines satisfaction of one or more criteria and determines a dry-eye condition if satisfaction is achieved. In one or more examples, a threshold number of dry-eye determinations are required for application of dry-eye mitigations such as warning notifications to the user of the electronic device. The threshold number of dry-eye determinations may change dynamically depending on factors such as environment, time (e.g., of day, week, etc.), and factors specific to the user. In one or more examples, dry-eye conditions vary depending on severity (e.g., not dry, slightly dry, moderately dry, severely dry, etc.). In one or more examples, the first or second ML model determine the severity of the dry-eye condition based on the predetermined one or more criteria. In some examples, the supervised ML model may include a neural network (e.g., deep learning model, logistic regression, linear or non-linear support vector machine, decision tree, random forest, recurrent neural network, transformer, boosted decision tree, convolutional neural network, gated recurrent network, long short-term memory network, etc.). In some examples, artificial intelligence (AI) or ML systems may utilize models that may be trained (e.g., supervised learning or unsupervised learning) using various training data, including data collected using a user device. Such use of user-collected data may be limited to operations on the user device. For example, the training of the model can be done locally on the user device so no part of the data is sent to another device. In other implementations, the training of the model can be performed using one or more other devices (e.g., server(s)) in addition to the user device but done in a privacy preserving manner, e.g., via multi-party computation as may be done cryptographically by secret sharing data or other means so that the user data is not leaked to the other devices. In some examples, in place of or in addition to support vector machines, one or a combination of many AI models (e.g., support vector machines, decision trees, random forests, neural networks, convolutional neural networks, recurrent neural networks, transformers) that can perform supervised machine learning may be used, including AI models not explicitly mentioned herein.

In one or more examples, the one or more supervised machine learning models include one or more support vector machines. In some examples, a support vector machine (SVM) is a ML algorithm used for the classification and outlier detection of data points within a feature space. SVM algorithms can find a hyperplane in an N-dimensional space that can separate data points in different classes in a feature space. The hyperplane of a 2-demensional space can be a line separating two classes or categories of vectors or data points. An optimal hyperplane in a 2-dimensional space is a line that maximizes a distance between the closest data points (or vectors) of different classes in the feature space. In some examples, a one-class SVM may be used as part of the ML models. A one-class SVM can use a kernel function to map input data to a higher-dimensional space where the data points are more separable. The one-class SVM can include a common kernel, such as a linear kernel, polynomial kernel 1, radial bases function (RBF) kernel, or a sigmoid kernel. The training process for building the distribution model can involve fitting the one-class SVM model to the threshold data points. An SVM algorithm can find an optimal hyperplane in an N-dimensional space that can separate data points in different classes in a feature space. The hyperplane of a 2-dimensional space can be a line separating two classes or categories of vectors or data points. In some examples, an optimal hyperplane in a 2-dimensional space is a line that maximizes a distance between the closest data points (or vectors) of different classes in the feature space.

In one or more examples, the one or more input devices include a visible light sensor, the electronic device further receives visible light imaging data from the visible light sensor while the visible light sensor is directed toward one or both eyes of the user of the electronic device. In some examples, the electronic device extracts one or more features from the received visible light imaging data. In some examples, the electronic device determines the dry-eye condition based on the one or more features extracted from the visible light imaging data. In some examples, the method optionally includes one or more visible light image sensors, such as charged coupled device (CCD) sensors, and/or complementary metal-oxide-semiconductor (CMOS) sensors operable to obtain images of the eye(s) of the user of the electronic device. Image sensor(s) also optionally include one or more cameras configured to capture movement of physical objects in the real-world environment. Image sensor(s) also optionally include one or more depth sensors configured to detect the distance of physical objects from electronic device. In some examples, information from one or more depth sensors can allow the device to sense and differentiate objects in the real-world environment from other objects in the real-world environment. In some examples, one or more depth sensors can allow the device to determine the texture and/or topography of objects in the real-world environment. In some examples, the data collected from the thermal image sensor(s) may be combined with the data collected from the visible image sensor(s) to produce electronic images blending the temperature and image data. In some examples, the blended temperature and image data includes a color map of the apparent temperature of the eye(s) of the user of the electronic device. The visible light sensor may be designed to capture high-quality images of the eyes under visible light conditions, providing information about the ocular surface and potential dry-eye conditions. The visible light sensor can be implemented using various technologies, such as charge-coupled devices (CCDs), complementary metal-oxide-semiconductor (CMOS) sensors, or CMOS image sensors with integrated optics. In some examples, the visible light sensor may be positioned on a head-mounted device, such as a headset, glasses, or goggles, allowing for targeting of the eyes and capturing high-resolution images of the ocular surface. In some examples, the visible light sensor can be integrated into a handheld device, such as a smartphone or tablets, which is then directed toward the eyes. In some examples, there may be more than one visible light sensor, such as a pair of sensors positioned on either side of the head-mounted device, allowing for stereo imaging and improved depth perception. In some examples, the visible light sensor may be combined with the thermal image sensor to achieve higher confidence in determining the dry-eye condition. In some examples, the visible light sensor may operate independently of the thermal image sensor. In some examples, the images captured by the visible light sensor may then be processed and analyzed to extract one or more features that may be indicative of dry-eye or other eye conditions, such as conjunctival or scleral redness, corneal staining, or tear film thickness. The information extracted from the visible light sensor may be used in conjunction with the thermal imaging data to provide a comprehensive assessment of the ocular surface and potential dry-eye conditions.

In one or more examples, the one or more features extracted from the received visible light imaging data include a characteristic of one or more blood vessels of one or both eyes of the user of the electronic device. The characteristic of the one or more blood vessels of one or both eyes may be any measurable property of the blood vessels, including but not limited to: diameter, length, tortuosity, branching pattern, red color intensity, blue color intensity, vessel-to-vessel distance, or any combination thereof. For example, the method may analyze the images captured by the visible light sensor to determine the red color intensity of the blood vessels in the conjunctiva or sclera, which can provide information about ocular surface health and potential dry-eye or other eye conditions. In some examples, the red color intensity is a measure of the amount of oxygenated hemoglobin present in the blood vessels, which can be indicative of changes in blood flow or ocular circulation. In some examples, the blue color intensity is a measure of the amount of deoxygenated hemoglobin present in the blood vessels, which can be indicative of changes in blood flow or ocular circulation. In some examples, the method may also analyze the blue color intensity to determine changes in deoxygenated hemoglobin levels, which can provide additional information about ocular circulation and potential dry-eye or other eye conditions and risks. In some examples, the vessel-to-vessel distance is a measure of the spacing between adjacent blood vessels, which can be indicative of changes in blood flow or ocular circulation. The method may also analyze the branching pattern of the blood vessels to determine changes in vessel diameter or ocular circulation. In some examples, the method may combine multiple characteristics, such as red color intensity and vessel-to-vessel distance, to provide an assessment of ocular surface health and potential dry-eye conditions. In some examples, the one or more blood vessels that are analyzed can include any of the major vessels in the eye, including but not limited to: the central retinal artery, the central retinal vein, the ophthalmic artery, the anterior ciliary arteries, or any combination thereof. The analysis of these blood vessels can provide insight into the health and function of the ocular circulation. In some examples, the method may also analyze the changes in the characteristic of the one or more blood vessels over time to detect changes that may be indicative of developing dry-eye conditions. For example, the method may track changes in the diameter of the blood vessels as a function of time, which can provide information about the integrity of the ocular circulation and potential dry-eye risks. For example, if the diameter of the blood vessels increases or decreases over time, this could indicate changes in the ocular circulation. In some examples, visible light imaging data may be extracted using a spectrophotometer. The spectrophotometer may extract the red color channels from one or both eyes of the user of the electronic device for the system to determine the redness of the sclera. The redness of the sclera may be used to determine dry-eye levels. In some examples, extracting the blood vessel dilation may include measuring the diameter of the blood vessels in the eye with the extracted imaging data and comparing to a baseline threshold determined for the user of the electronic device. An increase in diameter of the blood vessels may be an indication of dilation, which may contribute to determining dry-eye levels.

In one or more examples, the characteristic of one or more blood vessels of one or both eyes of the user includes a change of diameter over time for a blood vessel of one or both eyes of the user of the electronic device. A change of diameter over time may refer to the measurement of the variation in diameter or width of a blood vessel over a specific period of time. In some examples, machine learning methods such as edge detection may be used in conjunction with thresholding, morphological analysis, distance calculation (e.g., measuring the pixel distance between detected edges), or any other machine learning methods or combinations thereof to measure and calculate the diameter or width of a blood vessel, or any combination thereof. In the context of the method, this characteristic may be applied to the sclera blood vessels. The visible light sensor captures images of the eye at regular intervals, allowing for the measurement of changes in the diameter of the sclera blood vessels. In some examples, the diameter of the sclera blood vessels may be measured by the extracted features by extracting pixel width of the diameter of the sclera blood vessels from the image representations of the sclera blood vessels. In some examples, to measure the change in diameter, the method uses image processing techniques to extract the vessel profiles from the captured images. This may involve applying filters and thresholding techniques to enhance the contrast between the blood vessels and surrounding tissue. The resulting vessel profiles may then be analyzed to determine the diameter of each blood vessel. In some examples, the change in diameter over time is calculated by comparing the diameters of the same blood vessel at different time points. For example, if the method captures images of the eye every 10 minutes, the change in diameter could be calculated as the difference between the diameter measured at 10:00 AM and the diameter measured at 10:10 AM. In some examples, the visible light imaging data may be combined with the thermal imaging data to measure the diameter of the sclera blood vessels. In some examples, the measurement of the change in diameter over time for blood vessels provides information about the integrity of the ocular circulation and potential dry-eye or other eye risks. For example, an increase in the diameter of blood vessels in a short time (e.g., below a time threshold) may indicate vasodilation caused by inflammation of the eye due to a dry-eye condition.

In one or more examples, the one or more features extracted from the visible light imaging data include a redness of one or both eyes of the user of the electronic device. Measuring eye redness may refer to the process of determining the level of redness in the eyes of the user. In some examples, this is achieved by analyzing the visible light imaging data captured by the visible light sensor. The sensor may capture images of the eye under visible light conditions, allowing for the analysis of the color and reflectivity of the ocular surface. In some examples, to measure eye redness, the method uses image processing techniques to extract the intensity values of the red and green channels from the captured images. The intensity values can then be used to calculate a redness index, which is a numerical value that represents the level of redness in the eyes or parts of the eyes. In some examples, visible imaging data may be extracted using a spectrophotometer. The spectrophotometer may extract the red color channels from one or both eyes of the user of the electronic device for the system to determine the redness of the sclera. The redness of the sclera may be used to determine dry-eye levels. For example, increased redness in the eyes can be indicative of inflammation, irritation, or other conditions that may increase the risk of dry-eye.

In one or more examples, the electronic device, in response to determining a threshold number of determined dry-eye conditions have been met over a threshold period of time (e.g., a minute, an hour, etc.), provides one or more mitigations based on the dry-eye condition. In an example, the threshold number of determined dry-eye conditions is 5 determinations, 4 determinations, less than 4 determinations, or greater than 4 determinations. In one or more examples, setting a threshold number of dry-eye determinations before applying mitigations may reduce a possibility of false positive dry-eye determinations, where the system determines a dry-eye condition when one is not present. In one or more examples, dry-eye determinations are reduced to a threshold number when there is an increased rate of dry-eye determinations above a threshold number of times (e.g., 5 dry-eye determinations) in a threshold period of time (e.g., a minute, an hour, etc.). In one or more examples, reducing dry-eye determinations after exceeding a threshold number may reduce computational strain on the system, and optionally reduce the drain on a batter if one is present in the system. A mitigation may refer to any action or intervention taken in response to determining a dry-eye or risk of dry-eye condition, with the purpose of preventing dry-eye, alleviating the symptoms of dry-eye, reducing dry-eye severity, and preventing complications. Examples of mitigations may include providing warnings and alerts to the user to help them manage or prevent a dry-eye condition. Warnings and alerts may include information about treatments, therapies, or strategies that address the underlying causes of dry-eye, such as artificial tears, ointments, warm compresses, eye massage, humidity therapy, blinking exercises, avoiding irritants, using anti-inflammatory medications, and more. In some examples, warnings and alerts recommend the user to seek a healthcare professional. In some examples, the method may allow the user to or automatically schedule an appointment with the user's healthcare professional. In some examples, the system may send a blink reminder to remind the user to blink regularly or provide eye care reminders to take regular breaks from screen time, adjust display settings to reduce glare and blue light exposure, and avoid prolonged periods of inactivity. In some examples, the method may provide personalized recommendations for maintaining healthy eyes. In some examples, the system may track the user's dry-eye symptoms and provide insights on how to manage them, such as determining patterns of when and where dry-eye conditions occur. In some examples, the system may automatically adjust display settings, such as brightness and contrast, to reduce strain on the eyes and alleviate dry-eye symptoms. In some examples, the system may adjust brightness and contrast to modifying blinking behavior of the user, including inducing the user to blink more frequently to prevent or reduce dry-eye conditions by producing more tears. Additionally, the system may offer personalized coaching to help the user manage their dry-eye condition, such as providing tips for reducing screen time or adjusting the settings of the one or more displays of the electronic device.

In one or more examples, the one or more mitigations include: changing a speed of one or more fans of the electronic device. A fan may refer to a mechanical device that actively cools internal components by generating airflow. In some examples, the method may provide a mitigation that involves changing the speed of one or more fans of the electronic device. This may be done to modify blinking behavior of the user, including inducing blinking of the user of the electronic device, thereby increasing tear production helping to alleviate dry-eye conditions. In some examples, the method may reduce the fan speeds of the electronic device after determining a dry-eye condition to mitigate current dry-eye levels by reducing airflow to the eyes, thereby slowing tear evaporation. In some examples, this determination may be done by AI/ML systems that may utilize models that are trained (e.g., supervised learning or unsupervised learning) using various training data.

In one or more examples, the one or more mitigations include displaying, via the one or more displays, a notification to cease use of the electronic device. In some examples, warning the user includes a notification which includes: providing a notification based on the information, sending a message based on the information, displaying the information, controlling a user interface of an application based on the information, controlling a user interface of a health application and logging eye related health data of the user to the health application based on the information, controlling a focus mode based on the information, setting a reminder based on the information, or adding a calendar entry based on the information, or any combination thereof. In some examples, the notification may inform the user to apply a warm compress to the eyes. In some examples, warnings, alerts, and notifications may include information about treatments, therapies, or strategies that address the underlying causes of dry-eye, such as artificial tears, ointments, warm compresses, eye massage, humidity therapy, blinking exercises, avoiding irritants, using anti-inflammatory medications, and more. In some examples, warnings and alerts recommend the user to seek a healthcare professional. In some examples, the method may allow the user to or automatically schedule an appointment with the user's healthcare professional. In some examples, the system may send a blink reminder to remind the user to blink regularly or provide eye care reminders to take regular breaks from screen time, adjust display settings to reduce glare and blue light exposure, and avoid prolonged periods of inactivity. In some examples, the notification encourages ceasing use of the electronic device. For example, the system may display a notification on the one or more displays of the electronic device saying “Take a break! Your eyes need rest.” The notification may be designed to be attention-grabbing and easy to read and may include additional information or recommendations for reducing eye strain. In some examples, to further enhance the effectiveness of the notification, the system may also use haptic feedback and sounds to provide tactile sensation that grabs the user's attention. For example, the device may vibrate slightly to draw the user's attention to the notification or emit a gentle buzzing sound to alert them to take a break. In some examples, the notification combines different aspects such as haptic feedback and sound in a single action, encouraging the user more strongly. In some examples, the different varieties of notifications are displayed separately (e.g., only haptic, or only sound). In some examples, in addition to displaying a message, the system may also display or otherwise convey (e.g., by sound) specific information related to the user, such as blinking metrics or tear film quality. This may help users better understand their eye health and make informed decisions about how to manage a dry-eye condition. For example, the system may display a graph showing the typical blinking rate of the user or blinking rate at specific times (e.g., while using specific applications), with suggestions for improving the dry-eye condition. In some examples, notifications are customizable based on user preferences, such as tone pitch, vibration intensity, or notification frequency. In some examples, notifications are integrated with other applications to pause notifications and/or minimize screen time during extended use. In some examples, notifications may include schedules reminders for the user to take breaks and perform simple exercises, like rolling the eyes or looking away from the display. In some examples, dry-eye mitigation may be customizable and tailored to the individual needs and preferences of the user. In some examples, user adherence to dry-eye mitigation is tracked and rewards or other incentives provided for consistent compliance. Furthermore, in some examples, the system may provide additional resources or recommendations for reducing dry-eye condition, eye strain, or other eye conditions and promoting healthy eye habits. By providing these features, the system may help users develop healthy eye habits and reduce their risk of dry-eye and other eye problems. By taking proactive steps to manage their eye health, users can enjoy better overall health and well-being. In one or more examples of the disclosure, a method performed at an electronic device in communication with one or more displays and one or more input devices including one or more thermal image sensors configured to capture thermal imaging data of one or both eyes of a user of the electronic device: receiving the thermal imaging data of the user of the electronic device; extracting one or more features from the thermal imaging data; and in accordance with a determination that one or more criteria are satisfied, at least one criterion of the one or more criteria based on the one or more features extracted from the thermal imaging data, determining a dry-eye condition.

In one or more examples, the one or more features extracted from the thermal imaging data include spatial properties of tear film temperature regions of one or both eyes of the user of the electronic device.

In one or more examples, extracting spatial properties of tear film regions of one or both eyes of the user include segmenting the thermal imaging data to identify the one or more tear film temperature regions.

In one or more examples, the spatial properties include shape of the tear film regions.

In one or more examples, the spatial properties include size of the tear film regions.

In one or more examples, the one or more features extracted from the thermal imaging data include spatial properties of one or more temperature regions of the eye.

In one or more examples, the one or more features extracted from the thermal imaging data include a temperature change of one or both eyes of the user of the electronic device.

In one or more examples, the temperature change is a change in temperature over a predetermined duration measured from a first time after a blink to a second time, after the first time, while one or both eyes remain open.

In one or more examples, extracting one or more features from the thermal imaging data includes: identifying a first region of one or both eyes in the thermal imaging data, and extracting a temperature of the first region from the thermal imaging data.

In one or more examples, determining a dry eye condition comprises: applying one or more supervised machine learning models to the one or more features extracted from thermal imaging data.

In one or more examples, the one or more supervised machine learning models include one or more support vector machines.

In one or more examples, the one or more input devices include a visible light sensor, the method further comprising: receiving visible light imaging data from the visible light sensor while the visible light sensor is directed toward one or both eyes of the user of the electronic device, extracting one or more features from the received visible light imaging data, and determining the dry eye condition based on the one or more features extracted from the visible light imaging data.

In one or more examples, the one or more features extracted from the received visible light imaging data include a characteristic of one or more blood vessels of one or both eyes of the user of the electronic device.

In one or more examples, the characteristic of one or more blood vessels of one or both eyes of the user includes a change of diameter over time for a blood vessel of one or both eyes of the user of the electronic device.

In one or more examples, the one or more features extracted from the visible light imaging data include a redness of one or both eyes of the user of the electronic device.

In one or more examples, the method further comprises: in response to determining the dry eye condition, providing one or more mitigations based on the dry eye condition.

In or more examples, the one or more mitigations include: changing a speed of one or more fans of the electronic device.

In one or more examples, the one or more mitigations include displaying, via the one or more displays, a notification to cease use of the electronic device.

The present disclosure contemplates that in some examples, the data utilized may include personal information data that uniquely identifies or can be used to contact or locate a specific person. Such personal information data can include demographic data, content consumption activity, location-based data, telephone numbers, email addresses, twitter ID's, home addresses, data or records relating to a user's health or level of fitness (e.g., vital signs measurements, medication information, exercise information), date of birth, or any other identifying or personal information. Specifically, as described herein, one aspect of the present disclosure is tracking a user's biometric data.

The present disclosure recognizes that the use of such personal information data, in the present technology, can be used to the benefit of users. For example, personal information data may be used to display suggested text that changes based on changes in a user's biometric data. For example, the suggested text is updated based on changes to the user's age, height, weight, and/or health history.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates examples in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to enable recording of personal information data in a specific application (e.g., first application and/or second application). In addition to providing “opt in” and “opt out” options, the present disclosure contemplates providing notifications relating to the access or use of personal information. For instance, a user may be notified upon initiating collection that their personal information data will be accessed and then reminded again just before personal information data is accessed by the device(s).

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

What is claimed is:

1. A method comprising:
at an electronic device in communication with one or more displays and one or more input devices including one or more thermal image sensors configured to capture thermal imaging data of one or both eyes of a user of the electronic device:
receiving the thermal imaging data of the user of the electronic device;
extracting one or more features from the thermal imaging data; and
in accordance with a determination that one or more criteria are satisfied, at least one criterion of the one or more criteria based on the one or more features extracted from the thermal imaging data, determining a dry-eye condition.

2. The method of claim 1, wherein the one or more features extracted from the thermal imaging data include spatial properties of tear film temperature regions of one or both eyes of the user of the electronic device.

3. The method of claim 2, wherein extracting spatial properties of tear film regions of one or both eyes of the user includes segmenting the thermal imaging data to identify the one or more tear film temperature regions.

4. The method of claim 1, wherein the one or more features extracted from the thermal imaging data include a temperature change of one or both eyes of the user of the electronic device.

5. The method of claim 4, wherein the temperature change is a change in temperature over a predetermined duration measured from a first time after a blink to a second time, after the first time, while one or both eyes remain open.

6. The method of claim 1, the method further comprising:
in response to determining the dry-eye condition, providing one or more mitigations based on the dry-eye condition, wherein the one or more mitigations include displaying, via the one or more displays, a notification to cease use of the electronic device.

7. The method of claim 1, wherein the one or more input devices include a visible light sensor, the method further comprising:
receiving visible light imaging data from the visible light sensor while the visible light sensor is directed toward one or both eyes of the user of the electronic device;
extracting one or more features from the received visible light imaging data; and
determining the dry-eye condition based on the one or more features extracted from the visible light imaging data.

8. The method of claim 7, wherein the one or more features extracted from the received visible light imaging data include a characteristic of one or more blood vessels of one or both eyes of the user of the electronic device.

9. An electronic device, comprising:
one or more processors;
memory; and
one or more programs, wherein the one or more programs are stored in the memory and configured to be executed by the one or more processors, the one or more programs including instructions for performing a method comprising:
at the electronic device, in communication with one or more displays and one or more input devices including one or more thermal image sensors configured to capture thermal imaging data of one or both eyes of a user of the electronic device:
receiving the thermal imaging data of the user of the electronic device;
extracting one or more features from the thermal imaging data; and
in accordance with a determination that one or more criteria are satisfied, at least one criterion of the one or more criteria based on the one or more features extracted from the thermal imaging data, determining a dry-eye condition.

10. The electronic device of claim 9, wherein the one or more features extracted from the thermal imaging data include spatial properties of tear film regions of one or both eyes of the user of the electronic device.

11. The electronic device of claim 10, wherein extracting spatial properties of tear film regions of one or both eyes of the user includes segmenting the thermal imaging data to identify one or more tear film temperature regions.

12. The electronic device of claim 9, wherein the one or more features extracted from the thermal imaging data include a temperature change of one or both eyes of the user of the electronic device.

13. The electronic device of claim 12, wherein the temperature change is a change in temperature over a predetermined duration measured from a first time after a blink to a second time, after the first time, while one or both eyes remain open.

14. The electronic device of claim 9, the one or more programs further including instructions for:
in response to determining the dry-eye condition, providing one or more mitigations based on the dry-eye condition, wherein the one or more mitigations include displaying, via the one or more displays, a notification to cease use of the electronic device.

15. The electronic device of claim 9, wherein the one or more input devices include a visible light sensor, the one or more programs further including instructions for:
receiving visible light imaging data from the visible light sensor while the visible light sensor is directed toward one or both eyes of the user of the electronic device;
extracting one or more features from the received visible light imaging data; and
determining the dry-eye condition based on the one or more features extracted from the visible light imaging data.

16. The electronic device of claim 15, wherein the one or more features extracted from the received visible light imaging data include a characteristic of one or more blood vessels of one or both eyes of the user of the electronic device.

17. A non-transitory computer readable storage medium storing one or more programs, the one or more programs comprising instructions, which when executed by one or more processors of an electronic device, cause the electronic device to perform a method comprising:
at the electronic device, in communication with one or more displays and one or more input devices including one or more thermal image sensors configured to capture thermal imaging data of one or both eyes of a user of the electronic device:
receiving the thermal imaging data of the user of the electronic device;
extracting one or more features from the thermal imaging data; and
in accordance with a determination that one or more criteria are satisfied, at least one criterion of the one or more criteria based on the one or more features extracted from the thermal imaging data, determining a dry-eye condition.

18. The non-transitory computer readable storage medium of claim 17, wherein the one or more features extracted from the thermal imaging data include spatial properties of tear film temperature regions of one or both eyes of the user of the electronic device.

19. The non-transitory computer readable storage medium of claim 18, wherein extracting spatial properties of tear film regions of one or both eyes of the user includes segmenting the thermal imaging data to identify the one or more tear film temperature regions.

20. The non-transitory computer readable storage medium of claim 17, wherein the one or more features extracted from the thermal imaging data include a temperature change of one or both eyes of the user of the electronic device.

21. The non-transitory computer readable storage medium of claim 20, wherein the temperature change is a change in temperature over a predetermined duration measured from a first time after a blink to a second time, after the first time, while one or both eyes remain open.

22. The non-transitory computer readable storage medium of claim 17, the one or more programs further including instructions for:
in response to determining the dry-eye condition, providing one or more mitigations based on the dry-eye condition, wherein the one or more mitigations include displaying, via the one or more displays, a notification to cease use of the electronic device.

23. The non-transitory computer readable storage medium of claim 17, wherein the one or more input devices include a visible light sensor, the one or more programs further including instructions for:
receiving visible light imaging data from the visible light sensor while the visible light sensor is directed toward one or both eyes of the user of the electronic device;
extracting one or more features from the received visible light imaging data; and
determining the dry-eye condition based on the one or more features extracted from the visible light imaging data.

24. The non-transitory computer readable storage medium of claim 23, wherein the one or more features extracted from the received visible light imaging data include a characteristic of one or more blood vessels of one or both eyes of the user of the electronic device.
